(19) United States
(12) Patent Application Publication
(10) Pub. No.: US 2024/0212816 A1
(43) Pub. Date: Jun. 27, 2024

(54) ANALYZING GENOMICS DATA AND ANALYTICAL DATA

(71) Applicant: Cybele Microbiome, Inc., San Diego, CA (US)

(72) Inventors: Nicole M. Scott, San Diego, CA (US); James Lamoureux, San Diego, CA (US)

(21) Appl. No.: 18/288,626

(22) PCT Filed: Apr. 29, 2022
(86) PCT No.: PCT/US2022/027148

§ 371 Date: Oct. 27, 2023

Related U.S. Application Data
(60) Provisional application No. 63/181,821, filed on Apr. 29, 2021.

(51) Int. Cl. G16H 20/10; G16B 5/00; G16B 20/20; G16B 30/20; G16B 40/10

(52) U.S. Cl. CPC G16H 20/10; G16B 5/00; G16B 20/20; G16B 30/20; G16B 40/10

(57) ABSTRACT

In one or more implementations, genomics data and analytical data can be used to determine the presence of enzymes and organisms, such as bacteria, that may be present in an environment. The techniques described herein can determine candidate prebiotics that can be provided to an environment in order to generate postbiotics based on the presence of the enzymes and organisms.

### PRIORITY

This application claims the benefit of priority to U.S. Provisional Application Ser. No. 63/181,821, filed Apr. 29, 2021, which is incorporated by reference herein in its entirety.

### BACKGROUND

Genomics data and analytical data can be analyzed in various contexts to determine treatments for a number of biological conditions. It can often be challenging to bring together different types of genomics data and analytical data that is obtained from samples in order to arrive at results that are practically useful.

### BRIEF DESCRIPTION OF THE DRAWINGS

In the drawings, which are not necessarily drawn to scale, like numerals may describe similar components in different views. To easily identify the discussion of any particular element or act, the most significant digit or digits in a reference number refer to the figure number in which that element is first introduced. Some implementations are illustrated by way of example, and not limitation.

FIG. 1 illustrates flowcharts of processes to determine candidate prebiotics based on genomics data and analytical data.

FIG. 2 is a block diagram illustrating components of a machine, in the form of a computer system, that may read and execute instructions from one or more machine-readable media to perform any one or more methodologies described herein, in accordance with one or more example implementations.

FIG. 3 is block diagram illustrating a representative software architecture that may be used in conjunction with one or more hardware architectures described herein, in accordance with one or more example implementations.

FIG. 4 shows fold gene expression change for EC panD gene when Targeted Prebiotic for B-alanine (aTP) was added at different concentrations. E. coli culture was grown in LB until OD 1.5, control samples received additional growth media equivalent in volume to treatment cultures, which received growth media spiked with varying concentrations of targeted prebiotic in the amounts as indicated. Sampling of all cultures occurred at the start or time=0 (T0) as well as 1 (T1) and 3 hours (T3) post spike. Expression levels were measured through RNA and rtPCR. The gene for the turnover of aspartic acid to B-alanine (panD) shows a 2 fold increase in transcription versus control, indicating B-alanine metabolism pathway activation.

FIG. 5 shows several in silico predicted targeted prebiotics for healthy skin that were determined according to implementations herein.

FIG. 6 illustrates results from experiments where individuals N=2 were treated in 3 skin locations and in duplicate with both TP and carrier or carrier only (control). Prebiotics were no longer found on any site. None of the postbiotic ceramides were found on the control sites. Data are the result of swabs taken from these skin sites after 6 hours, extracted then run on Orbitrap (metabolomics).

FIG. 7 illustrates an example biochemistry platform as described here, consisting of in vitro, ex vivo, and in situ experiments and work, build evidence for safety, efficacy, mechanism and dosing of our targeted prebiotic compounds. An overview of the Bioinformatics platform is given below and also highlighted elsewhere.

FIG. 8 illustrates results from growth experiments, where empirical cultures grown overnight in LB broth are back diluted into new LB broth with varying concentrations of each compound so that the starting optical density (OD) at 600 nm is 0.05. Cultures are normally grown for 5 hours with shaking at 37° C., and samples taken for an OD₆₀₀ reading (FIG. 1). Longer growth experiments were also completed to examine the time of postbiotic production from 1 dose of TP.

FIG. 9 illustrates a Growth Curve Experiment Design. Cultures were grown for 5 hours or more, depending on the treatment, but OD₆₀₀ readings were done every hour to evaluate the growth rate of each culture.

FIG. 10 illustrates that Postbiotic Repellent compound is produced for at least 3 hours after the addition of the iTP. Example of postbiotic production after addition of Targeted Prebiotic. Here we show one demonstration of a predicted prebiotic input compound spiked into a mixed empirical skin culture sampled post-spike at 3 hrs for GCMS. The repellent output compound was found to be at levels that are higher than are necessary to result in repellency for Anopheles gambiae³.

FIG. 11 illustrates a compound Toxicity/Bacterial Cell Viability Assay. In order to test toxicity of prebiotics we added different concentrations to cultures. Samples were taken at several time points. Bacterial colony counts from the dilution plate used to determine viable cells (cells/mL).

FIG. 12 illustrates an example of safety and dosing viability test for the insect repellent Targeted Prebiotics (iTP). Here, mixed community microbiome cultures were grown overnight with different concentrations of predicted iTPs. In the experiment shown, 10 mM of TP input compound 2 was found to be too high of a dose and caused loss of viability in terms of colony forming units, or CFUs

FIG. 13 illustrates average microbial community growth with added concentration of Targeted Prebiotics (TP) for ceramides (c); here sphingosine and palmitic acid. From our mixed microbial culture collection (created from empirical skin microbiome samples), 3 microbiomes were grown in duplicate and spike-ins of the TP were added at different concentrations. Average of all values across experiments are given for time points along with standard error.

FIG. 14 illustrates a ceramide Standard Curve. ELISA against known concentrations of C-24 ceramides (x-axis) generate a standard curve.

FIG. 15 illustrates an example of generating M from KEGG. (A) shows a set of example reactions which are catalyzed by enzymes a-f. (B) the connectivity of the reactions in A), and (C) The connectivity matrix normalized such that all input compounds sum to 1 and outputs sum to −1

FIG. 16 illustrates that insect repellent compound is produced within 30 minutes and remains in culture at least 3 days. Bacterial cultures were given a single dose of input compound (FIG. 6) and samples were taken at various time points to determine how quickly repellent compound was produced and how long repellent compound was stable in culture, GC-MS. Due to space constraints, ceramides and hyaluronic acid data is not shown.

FIG. 17 illustrates that the ceramide Targeted Prebiotics (cTP) induce increased postbiotic ceramides with the microbiome in the presence of host cells. Production of postbiotic continues across 48 hours. Host-microbiome assays were conducted with 3 microbiome communities, where applicable. 1 dose at 0.02% of cTP was given for those applicable samples.

FIG. 18 illustrates that the ceramide Targeted Prebiotics (cTP) induce increased postbiotic ceramides with the microbiome in the host-microbiome assays. Carriers affect resulting postbiotic production. Host-microbiome assays were conducted with 3 microbiome communities, where applicable. 1 dose at 0.02% of cTP was given for those applicable samples. Formulation 2.0 contains cTP aka BioBloom™. “ambrosia” represents an ‘off the shelf’ well-known barrier and eczema cosmetic cream.

FIG. 19 illustrates an overview of assemblage models and the use of the platform to examine changes in prebiotics and postbiotic outputs.

FIG. 20 illustrates trans-well assay A.) Host-microbiome metabolite interaction can be studied in a tissue culture system in which human epithelial keratinocyte cells are plated in the lower chamber of a 6 well plate and then a 0.4 um membrane trans-well insert containing empirical microbiome samples are placed inside the well. B.) Microbiome and host cells share media and treatment conditions and are grown for 3 hours until sample harvest.

FIG. 21 illustrates that ceramide Targeted Prebiotics induce more ceramide postbiotics in the host-microbiome (trans-well) system than when added to human cells alone, or more than the host-microbiome alone. Postbiotic ceramide production from the trans-well assay is measured by ELISA. For each bar shown 3 different microbiome communities were used in triplicate experiments.

FIG. 22 illustrates that the ceramide Targeted Prebiotics (cTP) induce increased postbiotic ceramides with the microbiome in the host-microbiome assays. Carriers affect resulting postbiotic production. Host-microbiome assays were conducted with 3 microbiome communities, where applicable. 1 dose at 0.02% of cTP was given for those applicable samples. Formulation 2.0 contains cTP aka BioBloom™. “ambrosia” represents an ‘off the shelf’ well-known barrier and eczema cosmetic cream.

FIG. 23 illustrate host-microbiome assays A) supernatant is used for cytokines and cytotoxicity and B) cells ELISA and GCMS to examine postbiotic yield of ceramides

FIG. 24-26 illustrate results of cytokine markers show that ceramide Targeted Prebiotics reduce markers of sensitivity, irritation in the host-microbiome (trans-well system). Prior to each assay, 3 microbiome community cultures (IL-31, FIG. 24), (IL-1a, FIG. 25), and (IL-18, FIG. 26) were used and experiments were completed in triplicate.

FIG. 27 illustrates that targeted Prebiotic ceramides cause three long-chain high molecular weight ceramide postbiotics to be created directly on the skin. Individuals (N=2) were treated in 3 skin locations and in duplicate with both TP and carrier or carrier only (control). Data are the result of swabs taken from these skin sites after 6 hours, extracted then run on Orbitrap (metabolomics).

FIG. 28 illustrates abundances of several organisms from study participants' skin (n=42) before and after application of our cosmetic formulation with cTP, also known as BioBloom™. Participants were members of a 15 week IRB approved clinical trial.

FIG. 29 illustrates that the hyaluronic acid Targeted Prebiotics (hTP) induce increased postbiotic HA with the microbiome. Carriers affect resulting postbiotic production. Host-microbiome assays were conducted with 3 microbiome communities, where applicable. 1 dose at 0.02% of hTP was given for those applicable samples.

FIG. 30 shows results from host-microbiome (transwell) assays with hTP ELISA showing increased HA postbiotics in the presence of the microbiome. All assays were done in triplicate and for those using microbiome communities, N=3 communities were tested (also each in triplicate).

FIG. 31 illustrates ex vivo cytotoxicity experiments show that HA inputs (hTP) have less cellular toxicity than carriers or ingredients such as squalane. N=3 microbiome communities used where applicable, all experiments done in triplicate.

### DETAILED DESCRIPTION

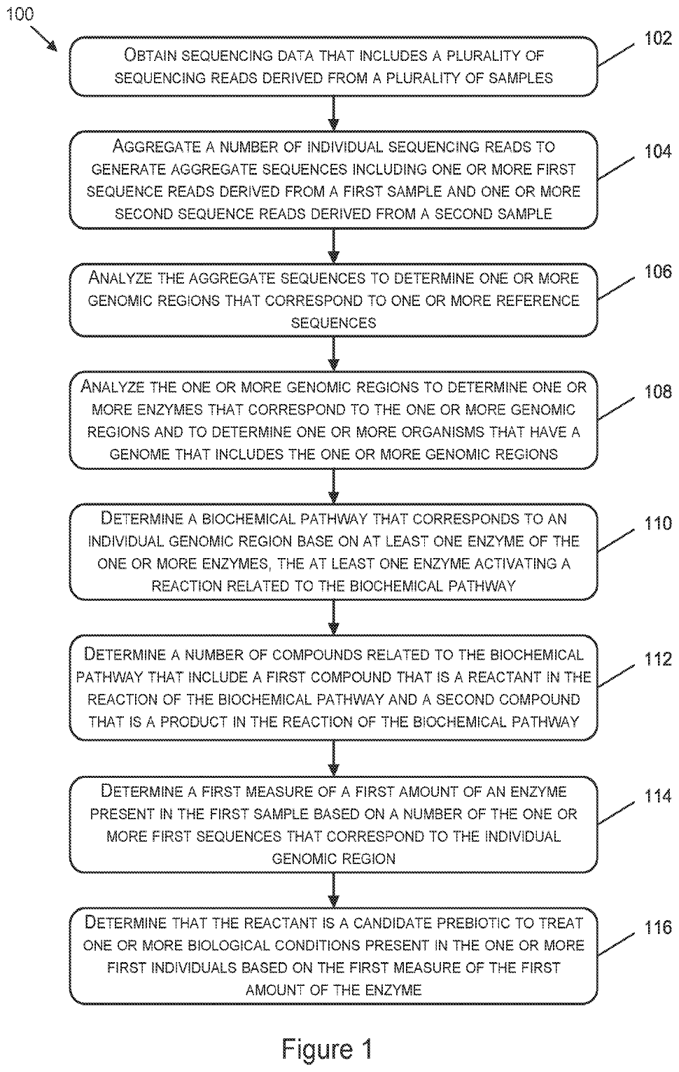

FIG. 1 illustrates flowcharts of processes 100 to determine candidate prebiotics based on genomics data and analytical data. The processes may be embodied in computer-readable instructions for execution by one or more processors such that the operations of the processes may be performed in part or in whole by the functional components of the environment 200 and system 300. Accordingly, the processes described below are by way of example with reference thereto, in some situations. However, in other implementations, at least some of the operations of the processes described with respect to FIG. 1 may be deployed on various other hardware configurations. The processes described with respect to FIG. 1 are therefore not intended to be limited to the environment 200 and the system 300 and can be implemented in whole, or in part, by one or more additional components. Although the described flowcharts can show operations as a sequential process, many of the operations can be performed in parallel or concurrently. In addition, the order of the operations may be re-arranged. A process is terminated when its operations are completed. A process may correspond to a method, a procedure, an algorithm, etc. The operations of methods may be performed in whole or in part, may be performed in conjunction with some or all of the operations in other methods, and may be performed by any number of different systems, such as the systems described herein, or any portion thereof, such as a processor included in any of the systems.

FIG. 1 is a flowchart illustrating example operations of a process 100 to determine candidate prebiotics based on genomics data and analytical data, according to one or more example implementations. At operation 102, the process 100 includes obtaining sequencing data that includes a plurality of sequencing reads. The plurality of sequencing reads can be derived from a plurality of samples.

The process 100 can also include, at operation 104, aggregating a number of individual sequencing reads of the plurality of sequencing reads to generate aggregate sequences. The aggregate sequences can include one or more first sequences of the plurality of sequences derived from a first sample of the plurality of samples obtained from a first individual and one or more second sequences of the plurality of sequences derived from a second sample of the plurality of samples obtained from a second individual.

At operation 106, the process 100 can include analyzing the one or more genomic regions to determine one or more enzymes that correspond to the one or more genomic regions. The one or more genomic regions can also be analyzed to determine one or more organisms having a respective genome that include the one or more genomic regions.

Additionally, at operation 108, the process 100 can include determining a biochemical pathway that corresponds to an individual genomic region of the one or more genomic regions based on at least one enzyme of the one or more enzymes that corresponds to the individual genomic region. The at least one enzyme can activate a reaction related to the biochemical pathway.

Further, the process 100 can include, at operation 110, determining a number of compounds related to the biochemical pathway. The number of compounds can include at least a first compound that is a reactant in the reaction of the biochemical pathway and a second compound that is a product in the reaction of the biochemical pathway.

At operation 112, the process 110 can include determining a first measure of a first amount of an enzyme of the one or more enzymes present in the first sample based on a number of the one or more first sequences that correspond to the individual genomic region.

The process 100 can also include, at operation 114, determining that the reactant is a candidate prebiotic to treat one or more biological conditions present in the one or more first individuals based on the first measure of the first amount of the enzyme.

In one or more examples, analytical data can be obtained from the first sample. The analytical data can be obtained using one or more analytical or biochemistry techniques, such as one or more mass spectrometry techniques, one or more liquid chromatography techniques, one or more thin layer chromatography techniques, or more gas chromatography techniques. In one or more additional examples, a first abundance of the reactant and a second abundance of the product can be determined in the sample based on the analytical data. In various examples, the reactant can be a candidate prebiotic based on the first abundance of the reactant and the second abundance of the product in the sample

In one or more examples, additional sequencing data can be obtained that includes a plurality of additional sequencing reads. The plurality of additional sequencing reads can be derived from a plurality of additional samples. The plurality of additional samples can include first additional samples that correspond to a first set of environmental conditions and second additional samples that correspond to a second set of environmental conditions. In various examples, a number of individual additional sequencing reads of the plurality of additional sequencing reads can be aggregated to generate additional aggregate sequences. The additional aggregate sequences can be analyzed to determine one or more additional genomic regions that correspond to the additional aggregate sequences. Further, the one or more additional genomic regions can be analyzed to determine one or more additional enzymes that correspond to the one or more additional genomic regions. The one or more additional genomic regions can also be analyzed to determine one or more additional organisms having a respective genome that includes the one or more additional genomic regions.

In various examples, based on the additional aggregate sequences first amounts of first enzymes present in a first additional sample can be determined. In addition, based on the additional aggregate sequences, second amounts of the first enzymes present in a second additional sample can be determined. Further, one or more differences between the first amounts and the second amounts can be determined based on the additional aggregate sequences.

In one or more examples, first additional analytical data can be obtained that is obtained from the first additional sample and second additional analytical data that is obtained from the second additional sample. Additionally, based on the first additional analytical data, a first additional abundance of the reactant can be determined. A first additional abundance of the product can also be determined. In various examples, based on the second additional analytical data, a second additional abundance of the reactant can be determined. Further, a second additional abundance of the product can be determined based on the second additional analytical data. In one or more examples, one or more first differences can be determined between the first additional abundance of the reactant and the second additional abundance of the reactant. Further, one or more second differences can be determined between the first additional abundance of the product and the second additional abundance of the product.

In various examples, based on the aggregate sequences, a plurality of organisms present in the first sample and the second sample can be determined. A subgroup of organisms included in the plurality of organisms can also be determined. The subgroup of organisms can correspond to a community of organisms that are of interest. In various examples, the subgroup of organisms can correspond to organisms that have at least a threshold abundance in one or more samples.

In one or more examples, first additional analytical data can be obtained that is derived from the first additional sample. Based on the first further analytical data, first additional measures of abundance for the subgroup of organisms in the first additional sample can be determined. Individual first additional measures of abundance can correspond to a respective first measure of abundance for an individual organism included in the subgroup of organisms. In addition, second further analytical data derived from the second additional sample can be obtained. In various examples, based on the second further analytical data, second additional measures of abundance for the subgroup of organisms in the second additional sample can be determined. Individual second additional measures of abundance can correspond to a respective second measure of abundance for an individual organism included in the subgroup of organisms. In one or more further examples, one or more differences can be determined between at least a portion of the first additional measures of abundance and at least a portion of the second additional measures of abundance.

In one or more examples, one or more correlations can be determined between at least one of the one or more first differences the one or more first differences between the first additional abundance of the reactant and the second additional abundance of the reactant or the one or more second differences between the first additional abundance of the product and the second additional abundance of the product. One or more additional correlations can also be determined between the one or more differences between at least a portion of the first additional measures of abundance and at least a portion of the second additional measures of abundance. In one or more illustrative examples, the one or more correlations are determined using one or more Bayesian network techniques.

In various examples, the first additional sample can be collected from a first environment that comprises a first formulation. The first formulation comprising a first amount of the reactant and a first carrier substance for the reactant. In addition, the second additional sample can collected from a second environment that comprises a second formulation. The second formulation comprising a second amount of the reactant and a second carrier substance for the reactant. In at least some examples, the first amount of the reactant can be different from the second amount of the reactant. In one or more additional examples, the first carrier substance for the reactant can be different from the second carrier substance for the reactant.

In one or more examples, one or more functions can be determined that can be executed to determine abundances of the subgroup of organisms. The one or more functions can be determined based on the first formulation and the second formulation. Additionally, the one or more functions can be determined based on the one or more differences between at least one of the one or more first differences the one or more first differences between the first additional abundance of the reactant and the second additional abundance of the reactant or the one or more second differences between the first additional abundance of the product and the second additional abundance of the product. Further, the one or more functions can be determined based on the one or more differences between at least a portion of the first additional measures of abundance and at least a portion of the second additional measures of abundance.

In one or more examples, a model can be generated that implements the one or more functions. The model can have a number of parameters that correspond to conditions within the first environment and the second environment. For example, at least one parameter of the number of parameters can correspond to an amount of prebiotic in a sample. At least one parameter of the one or more parameters can also correspond to a carrier in a formulation. In various examples, values of the conditions that correspond to the number of parameters can be obtained. At least a portion of the values of the conditions can be different from additional values of the conditions that correspond to the first environment and the second environment. Further, the model can be executed to determine abundances of at least a portion of the organisms included in the subgroup of organisms. The abundances can correspond to the values of the conditions. In one or more examples, the model can be generated using one or more artificial neural networks.

In one or more additional examples, one or more additional models can be generated that correspond to a simulated environment for one or more individuals. For example, a simulated environment for one or more phenotypes of individuals can be generated using empirical data. In various examples, genomics data, such as sequencing reads, and analytical data can be obtained from individuals in which a biological condition is present. The genomics and analytical data can be used to determine a simulated environment, such as a simulated skin microbiome that is present in individuals in which a biological condition is present, such as atopic dermatitis. In various examples, one or more additional models can be determined to simulate a skin microbiome of individuals based on samples obtained from a number of individuals in which one or more formulations were applied to the skin of the individuals. Genomic and/or analytical data can be obtained from the individuals to determine one or more parameters of the additional model. In at least some examples, the simulated environment represented by the additional model can be used to determine at least one of dosing information and/or carrier information that can result in maximizing activity of one or more biochemical pathways. In one or more illustrative examples, the one or more biochemical pathways can be activated to produce post-biotics that can treat the biological condition of the skin of individuals having the phenotype. In various illustrative examples, samples obtained from one or more individuals can be obtained and subjected to a number of experiments. The number of experiments can involve subjected the samples to environmental conditions that correspond to different doses of a candidate prebiotic and different carriers for the candidate prebiotic. In these scenarios, analytical data can be used to determine an amount of a postbiotic that is produced in relation to the different doses and formulations. The analytical data can be used to generate the one or more additional models that can then be used to predict the production of the post biotic with respect to additional dosing and/or carriers included in a formulation.

In various examples, the first sample can be obtained from skin of a first individual. In addition, the second sample can be obtained from skin of a second individual. The first individual can be included in a first phenotype. Further, the second individual can be included in a second phenotype. In one or more illustrative examples, the first phenotype can correspond to a presence of a biological condition with respect to individuals. The second phenotype can correspond to an absence of the biological condition with respect to individuals. In one or more additional illustrative examples, the biological condition corresponds to an abnormality related to skin of individuals.

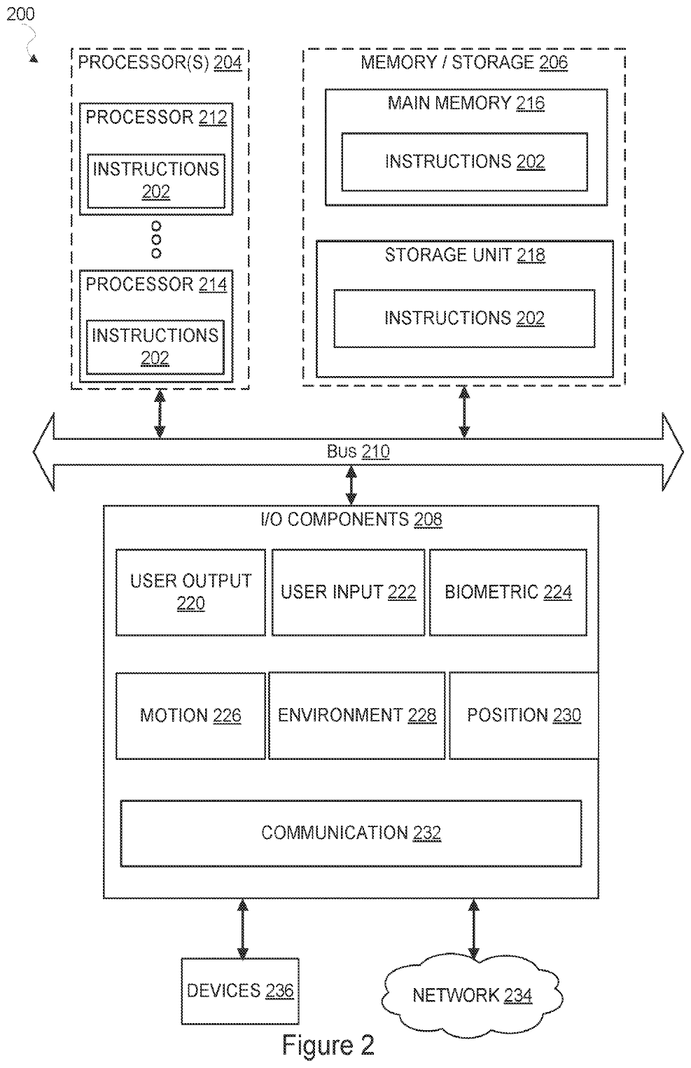

FIG. 2 is a block diagram illustrating components of a machine 200, according to some example implementations, able to read instructions from a machine-readable medium (e.g., a machine-readable storage medium) and perform any one or more of the methodologies discussed herein. Specifically, FIG. 2 shows a diagrammatic representation of the machine 200 in the example form of a computer system, within which instructions 202 (e.g., software, a program, an application, an applet, an app, or other executable code) for causing the machine 200 to perform any one or more of the methodologies discussed herein may be executed. As such, the instructions 202 may be used to implement modules or components described herein. The instructions 202 transform the general, non-programmed machine 200 into a particular machine 200 programmed to carry out the described and illustrated functions in the manner described. In alternative implementations, the machine 200 operates as a standalone device or may be coupled (e.g., networked) to other machines. In a networked deployment, the machine 200 may operate in the capacity of a server machine or a client machine in a server-client network environment, or as a peer machine in a peer-to-peer (or distributed) network environment. The machine 200 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, at network switch, a network bridge, or any machine capable of executing the instructions 202, sequentially or otherwise, that specify actions to be taken by machine 200. Further, while only a single machine 200 is illustrated, the term “machine” shall also be taken to include a collection of machines that individually or jointly execute the instructions 202 to perform any one or more of the methodologies discussed herein.

The machine 200 may include processors 204, memory/storage 206, and I/O components 208, which may be configured to communicate with each other such as via a bus 210. “Processor” in this context, refers to any circuit or virtual circuit (a physical circuit emulated by logic executing on an actual processor 204) that manipulates data values according to control signals (e.g., “commands,” “op codes,” “machine code,” etc.) and which produces corresponding output signals that are applied to operate a machine 200. In an example implementation, the processors 204 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) may include, for example, a processor 212 and a processor 214 that may execute the instructions 202. The term “processor” is intended to include multi-core processors 204 that may comprise two or more independent processors (sometimes referred to as “cores”) that may execute instructions 202 contemporaneously. Although FIG. 2 shows multiple processors 204, the machine 200 may include a single processor 212 with a single core, a single processor 212 with multiple cores (e.g., a multi-core processor), multiple processors 212, 214 with a single core, multiple processors 212, 214 with multiple cores, or any combination thereof.

The memory/storage 206 may include memory, such as a main memory 216, or other memory storage, and a storage unit 218, both accessible to the processors 204 such as via the bus 210. The storage unit 218 and main memory 216 store the instructions 202 embodying any one or more of the methodologies or functions described herein. The instructions 202 may also reside, completely or partially, within the main memory 216, within the storage unit 218, within at least one of the processors 204 (e.g., within the processor's cache memory), or any suitable combination thereof, during execution thereof by the machine 200. Accordingly, the main memory 216, the storage unit 218, and the memory of processors 204 are examples of machine-readable media. “Machine-readable media,” also referred to herein as “computer-readable storage media”, in this context, refers to a component, device, or other tangible media able to store instructions 202 and data temporarily or permanently and may include, but is not limited to, random-access memory (RAM), read-only memory (ROM), buffer memory, flash memory, optical media, magnetic media, cache memory, other types of storage (e.g., erasable programmable read-only memory (EEPROM)) and/or any suitable combination thereof. The term “machine-readable medium” may be taken to include a single medium or multiple media (e.g., a centralized or distributed database, or associated caches and servers) able to store instructions 202. The term “machine-readable medium” shall also be taken to include any medium, or combination of multiple media, that is capable of storing instructions 202 (e.g., code) for execution by a machine 200, such that the instructions 202, when executed by one or more processors 204 of the machine 200, cause the machine 200 to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

The I/O components 208 may include a wide variety of components to receive input, provide output, produce output, transmit information, exchange information, capture measurements, and so on. The specific I/O components 208 that are included in a particular machine 200 will depend on the type of machine. For example, portable machines such as mobile phones will likely include a touch input device or other such input mechanisms, while a headless server machine will likely not include such a touch input device. It will be appreciated that the I/O components 208 may include many other components that are not shown in FIG. 2. The I/O components 208 are grouped according to functionality merely for simplifying the following discussion and the grouping is in no way limiting. In various example implementations, the I/O components 208 may include user output components 220 and user input components 222. The user output components 220 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The user input components 222 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instrument), tactile input components (e.g., a physical button, a touch screen that provides location or force of touches or touch gestures, or other tactile input components), audio input components (e.g., a microphone), and the like.

In further example implementations, the I/O components 208 may include biometric components 224, motion components 226, environmental components 228, or position components 230 among a wide array of other components. For example, the biometric components 224 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 226 may include acceleration sensor components (e.g., accelerometer), gravitation sensor components, rotation sensor components (e.g., gyroscope), and so forth. The environmental components 228 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 230 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 208 may include communication components 232 operable to couple the machine 200 to a network 234 or devices 236. For example, the communication components 232 may include a network interface component or other suitable device to interface with the network 234. In further examples, communication components 232 may include wired communication components, wireless communication components, cellular communication components, near field communication (NFC) components, Bluetooth® components (e.g., Bluetooth® Low Energy), Wi-Fi® components, and other communication components to provide communication via other modalities. The devices 236 may be another machine 200 or any of a wide variety of peripheral devices (e.g., a peripheral device coupled via a USB).

Moreover, the communication components 232 may detect identifiers or include components operable to detect identifiers. For example, the communication components 232 may include radio frequency identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 232, such as location via Internet Protocol (IP) geo-location, location via Wi-Fi® signal triangulation, location via detecting an NFC beacon signal that may indicate a particular location, and so forth.

“Component,” in this context, refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example implementations, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an ASIC. A hardware component may also include programmable logic or circuitry that is temporarily configured by software to perform certain operations. For example, a hardware component may include software executed by a general-purpose processor 204 or other programmable processor. Once configured by such software, hardware components become specific machines (or specific components of a machine 200) uniquely tailored to perform the configured functions and are no longer general-purpose processors 204. It will be appreciated that the decision to implement a hardware component mechanically, in dedicated and permanently configured circuitry, or in temporarily configured circuitry (e.g., configured by software) may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering implementations in which hardware components are temporarily configured (e.g., programmed), each of the hardware components need not be configured or instantiated at any one instance in time. For example, where a hardware component comprises a general-purpose processor 204 configured by software to become a special-purpose processor, the general-purpose processor 204 may be configured as respectively different special-purpose processors (e.g., comprising different hardware components) at different times. Software accordingly configures a particular processor 212, 214 or processors 204, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled, components exist contemporaneously, Where multiple hardware communications may be achieved through signal transmission (e.g., over appropriate circuits and buses) between or among two or more of the hardware components. In implementations in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output.

Hardware components may also initiate communications with input or output devices, and can operate on a resource (e.g., a collection of information). The various operations of example methods described herein may be performed, at least partially, by one or more processors 204 that are temporarily configured (e.g., by software) or permanently configured to perform the relevant operations. Whether temporarily or permanently configured, such processors 204 may constitute processor-implemented components that operate to perform one or more operations or functions described herein. As used herein, “processor-implemented component” refers to a hardware component implemented using one or more processors 204. Similarly, the methods described herein may be at least partially processor-implemented, with a particular processor 212, 214 or processors 204 being an example of hardware. For example, at least some of the operations of a method may be performed by one or more processors 204 or processor-implemented components. Moreover, the one or more processors 204 may also operate to support performance of the relevant operations in a “cloud computing” environment or as a “software as a service” (SaaS). For example, at least some of the operations may be performed by a group of computers (as examples of machines 200 including processors 204), with these operations being accessible via a network 234 (e.g., the Internet) and via one or more appropriate interfaces (e.g., an API). The performance of certain of the operations may be distributed among the processors, not only residing within a single machine 200, but deployed across a number of machines. In some example implementations, the processors 204 or processor-implemented components may be located in a single geographic location (e.g., within a home environment, an office environment, or a server farm). In other example implementations, the processors 204 or processor-implemented components may be distributed across a number of geographic locations.

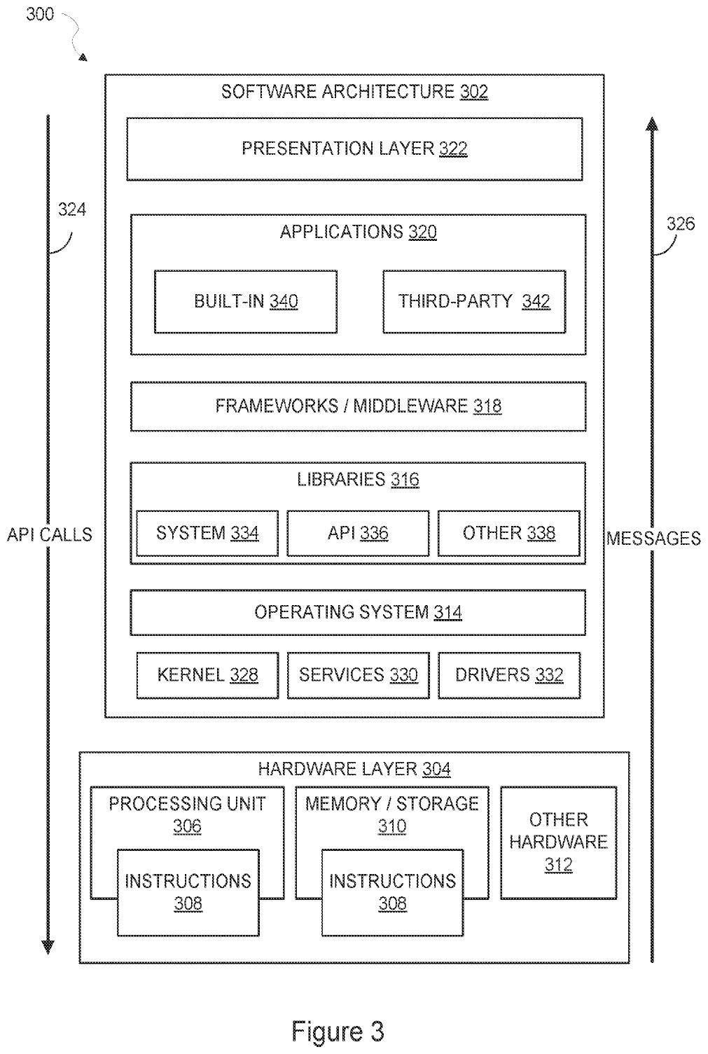

FIG. 3 is a block diagram illustrating system 300 that includes an example software architecture 302, which may be used in conjunction with various hardware architectures herein described. FIG. 3 is a non-limiting example of a software architecture and it will be appreciated that many other architectures may be implemented to facilitate the functionality described herein. The software architecture 302 may execute on hardware such as machine 200 of FIG. 2 that includes, among other things, processors 204, memory/storage 206, and input/output (I/O) components 208. A representative hardware layer 304 is illustrated and can represent, for example, the machine 200 of FIG. 2. The representative hardware layer 304 includes a processing unit 306 having associated executable instructions 308. Executable instructions 308 represent the executable instructions of the software architecture 302, including implementation of the methods, components, and so forth described herein. The hardware layer 304 also includes at least one of memory or storage modules memory/storage 310, which also have executable instructions 308. The hardware layer 304 may also comprise other hardware 312.

In the example architecture of FIG. 3, the software architecture 302 may be conceptualized as a stack of layers where each layer provides particular functionality. For example, the software architecture 302 may include layers such as an operating system 314, libraries 316, frameworks/middleware 318, applications 320, and a presentation layer 322. Operationally, the applications 320 or other components within the layers may invoke API calls 324 through the software stack and receive messages 326 in response to the API calls 324. The layers illustrated are representative in nature and not all software architectures have all layers. For example, some mobile or special purpose operating systems may not provide a frameworks/middleware 318, while others may provide such a layer. Other software architectures may include additional or different layers.

The operating system 314 may manage hardware resources and provide common services. The operating system 314 may include, for example, a kernel 328, services 330, and drivers 332. The kernel 328 may act as an abstraction layer between the hardware and the other software layers. For example, the kernel 328 may be responsible for memory management, processor management (e.g., scheduling), component management, networking, security settings, and so on. The services 330 may provide other common services for the other software layers. The drivers 332 are responsible for controlling or interfacing with the underlying hardware. For instance, the drivers 332 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 316 provide a common infrastructure that is used by at least one of the applications 320, other components, or layers. The libraries 316 provide functionality that allows other software components to perform tasks in an easier fashion than to interface directly with the underlying operating system 314 functionality (e.g., kernel 328, services 330, drivers 332). The libraries 316 may include system libraries 334 (e.g., C standard library) that may provide functions such as memory allocation functions, string manipulation functions, mathematical functions, and the like. In addition, the libraries 316 may include API libraries 336 such as media libraries (e.g., libraries to support presentation and manipulation of various media format such as MPEG4, H.264, MP3, AAC, AMR, JPG, PNG), graphics libraries (e.g., an OpenGL framework that may be used to render two-dimensional and three-dimensional in a graphic content on a display), database libraries (e.g., SQLite that may provide various relational database functions), web libraries (e.g., WebKit that may provide web browsing functionality), and the like. The libraries 316 may also include a wide variety of other libraries 338 to provide many other APIs to the applications 320 and other software components/modules.

The frameworks/middleware 318 (also sometimes referred to as middleware) provide a higher-level common infrastructure that may be used by the applications 320 or other software components/modules. For example, the frameworks/middleware 318 may provide various graphical user interface functions, high-level resource management, high-level location services, and so forth. The frameworks/middleware 318 may provide a broad spectrum of other APIs that may be utilized by the applications 320 or other software components/modules, some of which may be specific to a particular operating system 314 or platform.

The applications 320 include built-in applications 340 and third-party applications 342. Examples of representative built-in applications 340 may include, but are not limited to, a contacts application, a browser application, a book reader application, a location application, a media application, a messaging application, or a game application. Third-party applications 342 may include an application developed using the ANDROID™ or IOS™ software development kit (SDK) by an entity other than the vendor of the particular platform, and may be mobile software running on a mobile operating system such as IOS™, ANDROID™, WINDOWS® Phone, or other mobile operating systems. The third-party applications 342 may invoke the API calls 324 provided by the mobile operating system (such as operating system 314) to facilitate functionality described herein.

The applications 320 may use built-in operating system functions (e.g., kernel 328, services 330, drivers 332), libraries 316, and frameworks/middleware 318 to create UIs to interact with users of the system. Alternatively, or additionally, in some systems, interactions with a user may occur through a presentation layer, such as presentation layer 322. In these systems, the application/component “logic” can be separated from the aspects of the application/component that interact with a user.

Changes and modifications may be made to the disclosed implementations without departing from the scope of the present disclosure. These and other changes or modifications are intended to be included within the scope of the present disclosure, as expressed in the following claims.

### Example Implementations of the Disclosure

We have developed a paradigm method to identify, characterize, and utilize prebiotics to target and induce specific endogenous host skin microbiome metabolic pathways to create targeted postbiotics. Our insight was to focus on the redundant functions that exist in the microbiome rather than the taxonomic differences. Our deep knowledge of the microbiome and bioinformatics allowed us to develop this platform to parse large data sets and identify statistically significant prebiotic compounds that induce postbiotic compounds associated with healthy skin as well as other prebiotic compounds commercially relevant for insect repellency. After in silico identification, candidate prebiotic compounds were validated to produce the desired output postbiotic compounds through our experimental biochemistry platform, which has been optimized in a wet laboratory setting to measure candidate prebiotic and postbiotic compounds and our bioinformatic pipeline to examine microbial assemblages based on their specific chemical properties and integrate results from the experimental platform.

We have several assets that we have developed using our bioinformatic and biochemistry platforms. Specifically, we took a predicted (or in silico) candidate prebiotic and demonstrated we could indeed induce an insect repellent postbiotic in vitro and on human skin as a proof of concept. Using the same platforms we discovered prebiotics for ceramide postbiotics and also prebiotics for hyaluronic acid postbiotics (both ceramides and hyaluronic acid have major commercial relevance in skincare). Both ceramides and hyaluronic acid contribute to skin moisture and the skin barrier. We further validated the ceramides and hyaluronic acid using in vitro and in human experiments and showed that the postbiotics are produced at efficacious amounts.

Both our bioinformatic platform and biochemistry platform has been demonstrated in skincare. This space allows us to commercialize our technology faster. Our paradigm allows us to scale up quickly so that we can generate data for repeatability, applicability, and safety needed for commercialization of products faster than other methods of compound discovery. Our methods allow us to generate safety data necessary for the “new use of a common compound”. Our paradigm and platforms can be used in other environments such as the gut, oral health, reproductive system, companion animals, environmental systems, etc.

We validated the bioinformatics platform and pipeline to predict targeted prebiotics and their postbiotics

### Technical Objective 1.

Collected samples and metadata, classified phenotypes, extracted DNA, and created empirical skin microbiome cultures. We successfully:
Collected 45 skin swab samples from a number of diverse individuals for sequencing;
Extracted DNA of sufficient quality and quantity from the collected skin swabs;
Created 10 mixed culture communities.

### Technical Objective 2.

Demonstrated that microbiome communities can live in the presence of the predicted prebiotic input and postbiotic output compounds to assess toxicity and basic dosing. We successfully:
Showed that targeted prebiotics did not harm the skin microbiome in vitro;
Demonstrated that our prebiotic compounds could be safely fed to microbial communities in the range of 1 mM to 100 mM.

### Technical Objective 3.

Examined gene expression of metabolic pathways involving the in silico platform predicted prebiotic input and target postbiotic output compounds in vitro to address mechanism of action. We successfully:
Showed that the targeted prebiotic induced the genes necessary to convert the prebiotics into the postbiotic compounds;
Had a 2× increase in gene expression of target pathways when prebiotics were added, indicating pathway activation.

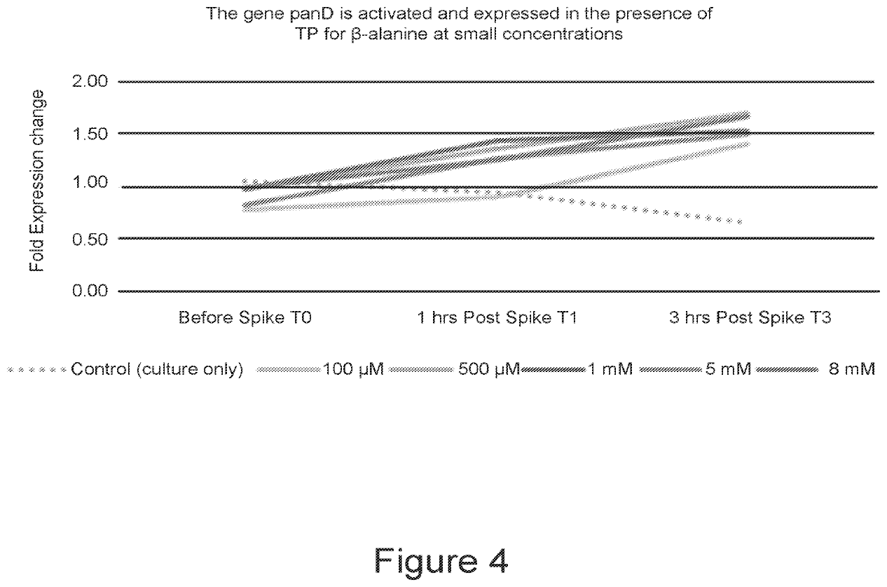

FIG. 4 illustrates fold gene expression change for EC panD gene when Targeted Prebiotic for B-alanine (aTP) was added at different concentrations. E. coli culture was grown in LB until OD 1.5, control samples received additional growth media equivalent in volume to treatment cultures, which received growth media spiked with varying concentrations of targeted prebiotic in the amounts as indicated. Sampling of all cultures occurred at the start or time=0 (T0) as well as 1 (T1) and 3 hours (T3) post spike. Expression levels were measured through RNA and rtPCR. The gene for the turnover of aspartic acid to B-alanine (panD) shows a 2 fold increase in transcription versus control, indicating B-alanine metabolism pathway activation.

### Technical Objective 4.

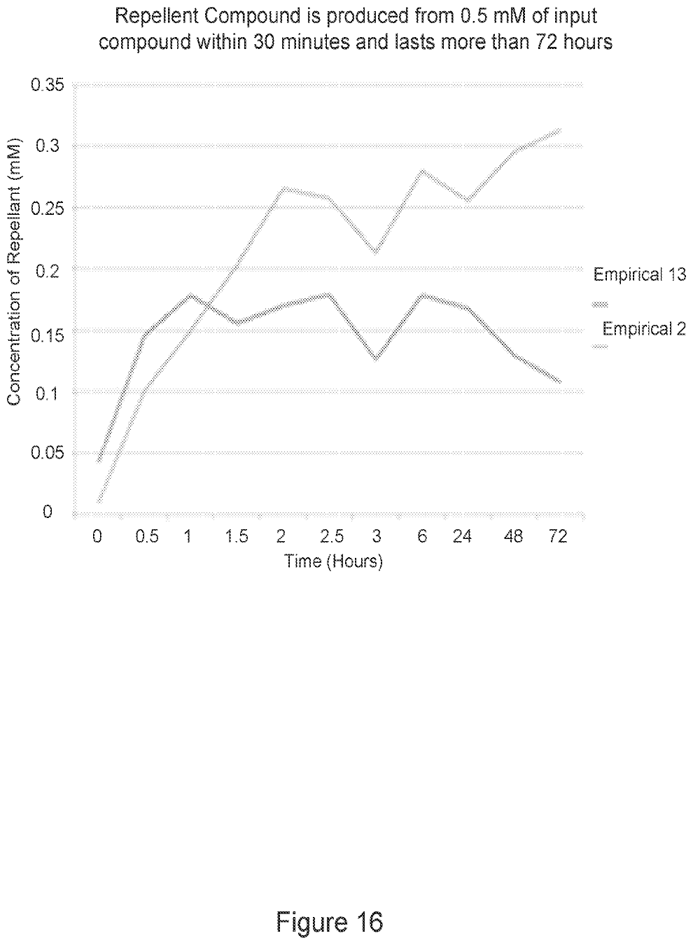

Assessed postbiotic output in vitro from targeted prebiotics. We successfully:
Confirmed that our platform predicted targeted prebiotics and postbiotics perform as predicted in vitro
Determined the best methods for detection and quantification of our lead predicted postbiotic output compounds: 2-phenylethanol, ceramide, and hyaluronic acid
Developed, baselined, and optimized methods for measuring ceramide and hyaluronic acid pathway output compounds in vitro
Validated that postbiotic output compounds were produced in vitro (via GC-MS and ELISA) at clinically relevant thresholds
Showed that on average production time of output compounds started at 30 minutes and was still detectable in clinically relevant amounts for 48 hours (FIG. 16)

### Technical Objective 5.

Assessed composition and function of microbial communities treated with predicted prebiotic input compounds in vitro. We used metagenomics to study the changes in the communities after additions of predicted input compounds.

We successfully:

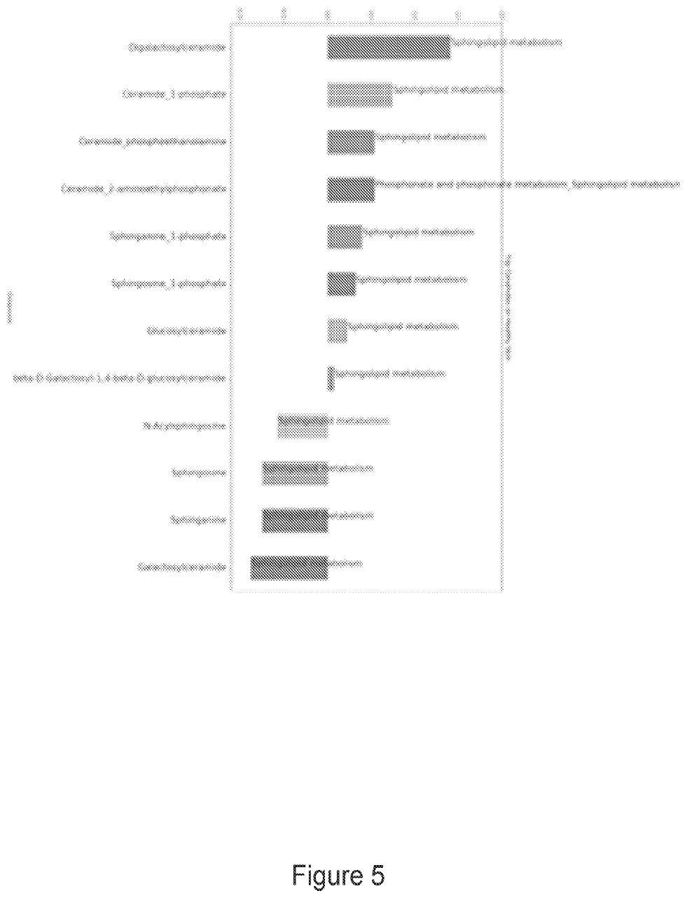

Consistently extracted sufficient amounts of non-degraded microbial DNA from samples for sequencing with an average 14.66 ng/μL, well above the 2 ng/μL needed for shotgun sequencing:
Completed quality control, annotation, processing, and basic analyses of genetic sequencing data;
Used high-throughput next generation DNA sequencing: annotation and assembly of the data to generate 170M DNA sequences for analysis (mean 9M sequences/sample);
Examined diversity within and between samples;
Showed that our prebiotics did not alter microbiome community structure;
Demonstrated that despite differences in composition diversity (taxonomy differences) across samples, adding prebiotic inputs did not shift diversity within samples (N=3, triplicate);
Showed that prebiotic inputs caused targeted functional changes in vitro;
Example platform methods predicted input and output compounds as shown in FIG. 5 and found 5 high probability candidate prebiotic input compounds that would create ceramide and 6 compounds that would create hyaluronic acid postbiotics;
Validated that previously predicted prebiotic input and postbiotic output compounds and their metabolic pathway genes were indeed different in the treated community assemblages (N=3) and found 4 genes whose metabolite scores were statistically significant (p<0.05) between groups (atopic dermatitis vs normal skin).

### Goal 2.

Demonstrated that predicted target prebiotics induced specific pathways and create predicted output compounds in situ on human skin.

### Technical Objective 6.

Assessed and validated that methods can be used on the human skin to induce the native microbial communities to create the predicted output compounds

We successfully:
Showed postbiotic output increases when the prebiotic was applied to human skin;
Obtained quantitative abundances of candidate postbiotic output compounds in situ;
Demonstrated that our input prebiotic compounds induced 210× output postbiotic compounds compared to controls on human skin.

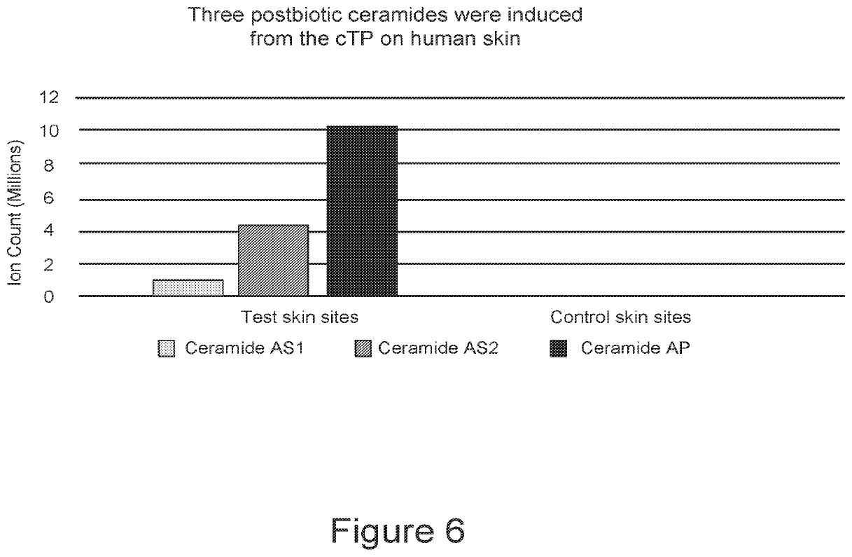

FIG. 6 shows that individuals N=2 were treated in 3 skin locations and in duplicate with both TP and carrier or carrier only (control). Prebiotics were no longer found on any site. None of the postbiotic ceramides were found on the control sites. Data are the result of swabs taken from these skin sites after 6 hours, extracted then run on Orbitrap (metabolomics). See also G2TO6

### Part 2. Technical Objectives, Approach and Work

We focus first on the prebiotics that induce ceramide postbiotics, as these ingredients and their associated developed formulation. Ceramides are crucial in skin health—regulating key processes such as cell differentiation, cellular proliferation, and cell death. Ceramides are a major contributor to the outer ‘skin barrier’ of the skin and are known to decline after the age of 20. The loss of the skin barrier is a known precursor to skin disease, including atopic dermatitis, eczema, and psoriasis.

Since we have a ‘new use of a common compound’ for our ceramide prebiotics, these data generated as part of our work—from in vitro, ex vivo and human work are necessary for building our repertoire of safety data for commercialization and claims.

This platform show the applicability and repeatability of our prebiotics, dosing, and formulation. We use our paradigm including the bioinformatic platform and biochemistry platform to demonstrate safety, dosing, formulation, effectiveness.

We have evidence for our targeted prebiotics to create the desired postbiotics with our insect repellent, ceramide and hyaluronic acid assets. In the instance of the cTP we have evidence of both cTP and its accompanying carriers (formulation) is efficacious on the skin of a broad range of individuals.

### Part 2.1 the Biochemistry Platform

### G1: Assess Repeatability, Dosing, and Create a Formulation

Here we build upon our platform developed in our work and assess our targeted prebiotics that create ceramide postbiotics for repeatability and reliability, dosing, and effectiveness across a large set of diverse skin microbiomes in vitro. We also screen a set of carriers for a basic formulation and develop in silico models which will help in reducing experimental parameters in vitro, ex vivo, and in situ experiments.

### G2: Assure Applicability and Safety of the Prebiotics Ingredients Ex Vivo and In Vivo

We scale up our recently developed host-microbiome system. We show this system with examples from the ceramide targeted prebiotics. With this system we assess carriers and test for basic safety. Using metabolomics we assess for the production of our ceramide postbiotics on a variety of diverse human skin types.

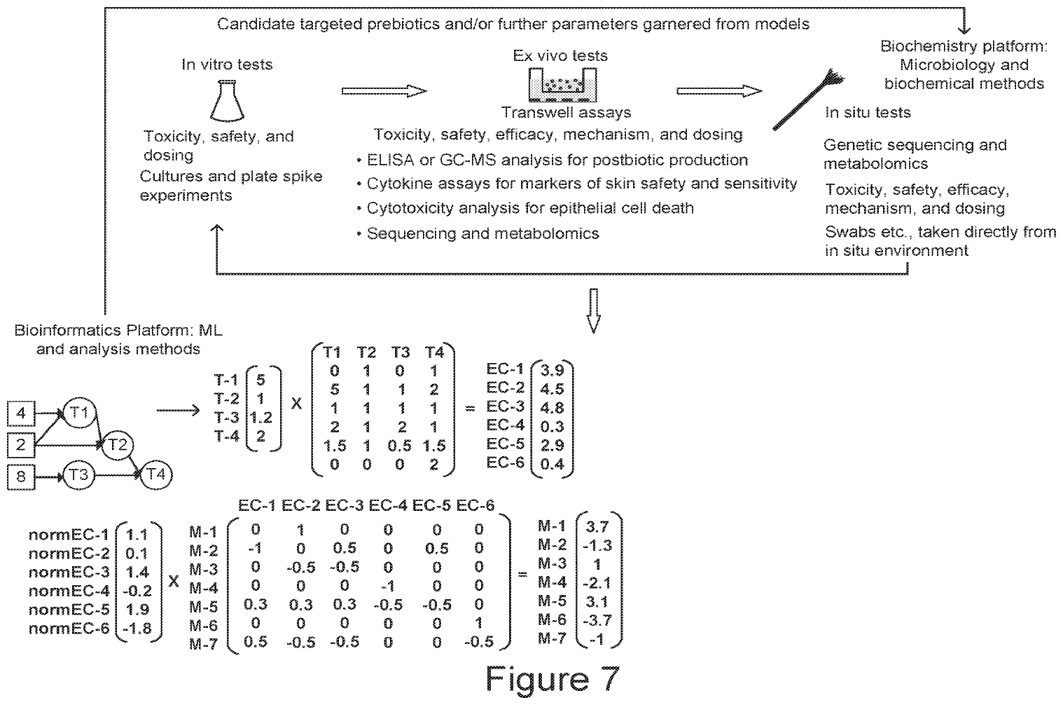

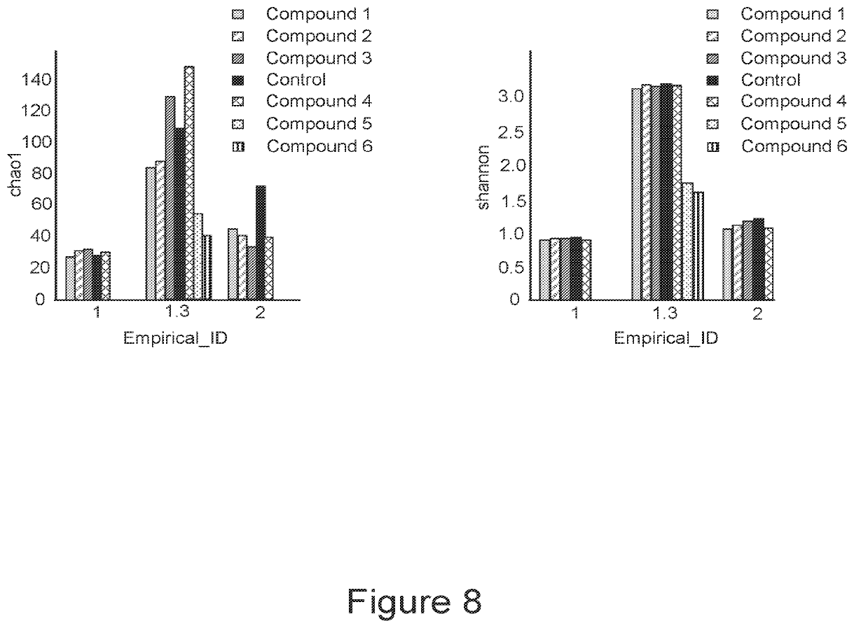

The biochemistry platform as shown in FIG. 7, consists of in vitro, ex vivo, and in situ experiments and work, build evidence for safety, efficacy, mechanism and dosing of our targeted prebiotic compounds. An overview of the Bioinformatics platform is given below and also highlighted elsewhere. In FIG. 7, dashed arrows indicate initial steps to find candidates using prediction methods. Candidates are then screened through biochemistry platform. Biochemistry results feed into the making models, which then predict core constituents and their functions. These models define key organisms and their parameters and help to shape formulations and experiments.
G3: Demonstrated scalability of our platforms and applicability of platform methods as described in G1 and G2 by predicting targeted prebiotics for hyaluronic acid (HA), an important ingredient in skincare health, and then using our biochemistry platform top show safety, efficacy and developed a basic cosmetic formulation.

Our bioinformatic platform predicts the prebiotics and postbiotic compounds and then our biochemistry platform tests for their applicability and safety both in vitro (G1), ex vivo/in vivo (G2) allowing us to complete a basic set of carriers (formulation).

### Part 2.2 Technical Overview

### G1TO1 Baseline Skin Microbiome and Metabolomics and Create a Diverse Skin Microbiome Culture Collection

We create a diverse empirical skin microbiome culture collection from facial skin swab samples collected from 51 individuals of different ethnicities and ages from an ongoing clinical study. We also extract DNA and complete genetic shotgun sequencing and metabolomic from skin swabs to establish baselines.

### G1TO2 Assess Applicability of Prebiotic Dosing and Postbiotic Output Across Skin Culture Collection In Vitro

In our previous work we demonstrated the proof-of-concept work that our platform did indeed identify mosquito repellent prebiotics, prebiotics for ceramides, and prebiotics for hyaluronic acid, that could be used ultimately, in situ, to make their respective postbiotics. Our work was limited to a small set of empirical skin microbiomes (N=10) and samples. To confirm that our future skincare prebiotics are commercially viable and safe: we examine ceramide prebiotic dosing across our diverse created skin cultures (N=51) from TO1. We also examine safety and dosing on postbiotic ceramide production using viability/growth assays, and for a subset of cultures, using metagenomics and metabolomics. We also compare single dosing vs multiple dosing profiles.

G1TO3 Assess Carriers for Our Prebiotics and their Effects on Various Empirically Derived Microbiome Cultures

We assess a set of carriers for our prebiotics and their effects on various empirically derived microbiome cultures from TO1. We also develop in silico models that aid us in reducing the parameter space for testing. This helps us create formulations that can be tested ex vivo (TO4, TO5, TO6) and aids in creating a final cosmetic formulation for our commercial pursuit.

### G2TO4 Assessing Formulation Dosing and Postbiotics Consistency Ex Vivo

We develop a host-microbiome ex vivo system to assess our in vitro findings (TO2) for translation into an ex vivo system. This gives us the opportunity to directly examine the ceramide prebiotics effects on microbiomes and the accumulation of postbiotic ceramides with the skin. We assess repeatability of our prebiotics in this system across our culture collection (TO1) to show applicability.

### G2TO5 Assess Markers of Safety Using Ex Vivo Host-Microbiome System

To commercialize our prebiotics we must assure safety. We assessed a number of markers of safety in our host-microbiome assay system (TO4) including irritation, sensitivity, cell-health and cell death in our carriers (TO3) with our prebiotics for postbiotic ceramide production. Previously, we were able to detect a robust postbiotic ceramide output after only 30 minutes of input probiotic addition to skin microbiome cultures in vitro. The postbiotic ceramides were also continuously detected up to 72 hours later. Here we measured the postbiotic across time through the use of the ex vivo system (TO4). We examined both output production onset and output half-life stability and repeatability across samples and individuals across time.

### G2TO6 Assess the Postbiotic Ceramide Production on Human Facial Skin.

We used metabolomics to assess the postbiotic ceramide production from human skin in our on-going clinical study. The primary drivers here are additional commercial safety and basic formulation stability for effective delivery and administration of our products.

### Stretch G3TO7 Assess Applicability of Hyaluronic Acid (HA) Prebiotic Postbiotic In Vitro and Ex Vivo

Here we used the scaled up in vitro and ex vivo experiments from TO3 and TO5 with our hyaluronic acid prebiotics to examine the applicability to diverse skin microbiome communities. We again built models using metagenomic sequencing and metabolomics (TO3) to aid in our lab experiments.

### Stretch G3TO8—Assess Applicability of Hyaluronic Acid (HA) In Situ

Using methods designed in TO6, we examine postbiotic hyaluronic acid production on human skin, using our on-going clinical study of the human skin microbiome.

### 2.3 Approach and Examples

### TO1 Baselined Skin Microbiome and Metabolomics and Created a Diverse Skin Microbiome Culture Collection

We used skin swab samples previously collected from 51 diverse individuals of different ethnicities and ages from an ongoing clinical study. We cultured these samples for experiments assessing repeatability using metagenomics and metabolomics. We also directly sequenced these swabs as part of TO1. The methods for sample processing for shotgun sequencing and metabolomics will be used as methods of examining metabolisms (genes, organisms, metabolic pathways) in culturing and spike experiments.

### Baselined Skin Sample Collection and Sample Size:

We carried out an ongoing longitudinal clinical skincare study (Integreview IRB #Beta2.0)-01). After prior consent we collected a set of baseline skin swabs for microbiome and metabolomic samples from 51 people enrolled in the study. Sampling was performed at 1 in×1 in face skin areas for approximately 10 seconds, with pre-moistened swabs at each site in 50:50 ethanol/water for Mass Spec (MS) analysis (metabolomics) or 50 mM Tris pH 7.6, 1 mM EDTA, and 0.5% Tween 20 for nucleic acid analysis (microbiome). Swabs were labeled and stored at −80° ° C. until use. Additionally, all basic demographics including age, ethnic origin, and sex have been collected. We followed Minimum Information about any (x) Sequence checklists (MIxS) that were established to store metadata for these samples. This enabled the processing and analysis of these samples, which is crucial in furthering partnerships with strategic investors and investors. The sample numbers for the study were based on current resources and calculations based on effect size in previous studies. We have measured effect sizes for markers of skin inflammation on the order of 20-30% differences. Due to this our aim was to have at least 15 individuals in each skin group subclass (sensitive, non-sensitive/normal) for adequate statistical power (StatMate, based on effect size from and 2 subclasses in our own recent proprietary clinical study). We currently have 51 individuals enrolled, N=17 with sensitive skin and N=34 with non-sensitive skin and will continue to recruit individuals.

Given that we are interested in the redundant functional processes in the skin microbiome—we collected samples based on the self-reported phenotype of skin sensitivity—we collected from persons age 18 and older and did not restrict ourselves to collecting samples from individuals based on sex or ethnicity. The study currently includes individuals 18-74 in age, males (N=10) and females (N=41), of diverse race and ethnicities (American Indian or Alaska Native (N=1), Asian (N=7), Black or African American (N=3), and White (N=40)). We continue to recruit additional individuals to increase the size and diversity of the cohort.

### Created a Diverse Invitro Empirical Microbiome Culture Collection:

Skin swabs were inoculated in Luria Bertani (LB) broth, which is a standard rich culture media, then were grown at 37° C. with shaking. For all cultures, after growth to late log phase, 1 ml samples of culture were mixed with 1 ml of 50% glycerol, and frozen at −80° C. for later experimental use.

### Baselined Microbiome and Metabolome Skin Samples:

Samples collected directly from skin were processed for metagenomic shotgun sequencing and metabolomics. Shotgun sequencing of skin swabs prior to culturing provides a qualitative snapshot of the ‘core’ microbiome and ‘core’ functional processes and as baselines, they allow us to examine losses incurred on culturing (TO2, TO3). We examined diversity of microbiomes among other demographics and skin types (sensitive and non-sensitive). We clustered samples based on microbial diversity, least to most diverse based on several metrics, and subsampled for cultures based on these results in order to keep our processing and experiments efficient.

### Sample Processing, Library Preparation and Sequencing:

Note these methods are examples of applied methods of sample handling and preparation.

A brief overview of the protocols is included here. Microbiome swabs will be extracted using QIAamp DNA Microbiome Kit with several modifications to increase lysis. Although this kit depletes host DNA, we are aware that computational methods and deeper sequencing are needed to reach low abundance microbes in the samples. Using the extracted DNA from skin samples, libraries will be made using the Kapa HyperPlus® kit, (Roche) and the Illumina HiSeq®-2500 platform. We will choose 151 bp paired end sequencing and an insert size of 350 bp for sequencing. We aim for 2M reads per sample—a number based on our previous work that is necessary and sufficient for obtaining compound targets for our pipeline and methods. We also include 3 sample and 3 library prep replicates (from a single sample) per lane to assess quality control and technical variation. Replicate samples are sequenced separately and in different lanes.

### Metagenomics Analysis and Methods:

These Methods were Commonly Applied to all Sequenced Samples and have been Integrated into an Internal Pipeline we have Built.

Our methods will require both assembly and direct database annotation. To begin, we preprocess sequences including removing cloning vector sequences, quality trimming to remove low-quality bases, and screening to remove verifiable sequence contaminants. The assembly of these data without vector trimming can produce chimeric contigs where the vector sequence, being common to most reads, draws together unrelated sequence.

### Assembly and Annotation:

For draft genome assembly metaSPAdes will be used, this employs “efficient assembly graph processing” that utilizes rare variants and includes error-correcting, it is based on SPAdes. For each scaffold, we will determine properties such as the GC content, coverage, genetic code, and profile of phylogenetic affiliation based on the best match for each gene in Uniref90. On the basis of analyses of these data, as well as emergent self-organizing map (ESOM)-based analyses of tetranucleotide frequencies and time series relative abundance draft genomes will be generated that will include scaffolds from multiple samples. Scaffolds for the same genome found in different samples will be aligned to yield longer fragments, leveraging the observation that fragmentation of assemblies is dependent on the context (community composition). We will use Bowtie for read mapping. Paired-read information will be used to extend and join contigs and to fill in gaps by the assembler. The advantage to assembly-based methods is that functional attributes can be more directly linked to organism context.

### Direct Annotation:

While assembly is a useful method for sample composition, we also note that it limits the ability to examine low abundance microbes that could be suppressed. Because the goal of this aim is to understand the components necessarily driving the functional differences in the community, we also will directly annotate genes for function. Because we will be utilizing samples from human skin, we also benefit from the amount of public data and databases that exist with annotated microbiome data that were largely formed to study human-associated organisms. To do this we will use alignment to reference genomes using shotgun community profiling, MetaPhlAn and Centrifuge for read-mapping, and with additional functional abundance annotations from HUMAnN2. Enzyme Commission (EC) abundances will be gathered from the functional abundances, quantile normalized and then log 2 transformed before platform analysis. We expect that ORFans—sequences that do not annotate to any reference sequence, to be rarer due to factors including erroneous protein coding calls for sequence, true novelty, or genetic heterogeneity.

### Metabolomics Analysis and Methods:

We preform targeted and untargeted metabolomics and cheminformatics on samples. Swab samples taken from skin are extracted at 50% EtOH and analyzed using LCMS. A reverse phase gradient on a C18 column will be used for chromatography and molecules analyzed with a high resolution Orbitrap mass spectrometer run in an untargeted fashion. Each samples' data was analyzed with MZmine to determine the features and relative quantifications. Detected features are searched against all public spectral libraries available for LCMS data and a reference library of compounds from related studies²⁹⁻³¹. Calculated and reported retention indices and injection of authentic synthetic reference compounds will provide additional information for identification. These methods provide a baseline for ceramides and associated compounds on the skin. Also, overlaying the observed ceramides with a pathway enrichment analysis (from annotated sequence data) should allow us to bin biochemical pathways most associated with sensitive skin microbiomes and those most associated with skin barrier and ceramides to examine any off-target effects important in safety.

Research has shown that the skin microbiome's genome size has a large variance, but an average genome size of 5.5 kb, with ˜2M per sample, these sequencing data should be adequate with direct annotation techniques. These data, though interesting to compare among themselves (sensitive skin vs non-sensitive skin phenotypes for example) and also serve as baselines and comparisons in experiments in TO2, TO3, TO4, and TO5. Due to assembly-based methods and additional ability to identify functional genes, related pathways, and organisms, our methods are less constrained by ‘known’ metabolisms and pathways, and can be used to find new previously unknown candidate prebiotics and postbiotics, metabolites, especially in cases where we do not have a-priori information about biological and functional relationships to phenotype. For skin conditions, such as atopic dermatitis (AD), eczema, and psoriasis—we expected to see increases in ceramide and ceramide-related pathways. Indeed our candidate prebiotics and postbiotics were statistically significantly changed in these functional pathways between those without atopic dermatitis and those with AD. Phenotypic clustering based on the metabolomics and binning biochemical pathways will co-localize additional previously unknown associated skin inflammation metabolites compounds (by showing statistical differences between groups) and in turn compounds that can be used to induce these metabolites in future work. We anticipate—based on the effect size from a previous clinical study—that we will need to sequence <50 individual subjects to have power to detect a difference between sensitive skin groups (2 groups), although we acknowledge that due to the complexity and multiple compounds being tested, additional samples may be required. Though we actively collect sample swabs from the face skin (and those used in previous studies) we also collect samples from additional non-standard sites (such as the arm). If we do not see meaningful statistical differences between the groups, we can easily collect additional samples. It is also possible that our samples show less diversity than would be expected—or that we have less power than we anticipated-however, we continue to collect samples through our study to increase sample sizes, diversity, and power. Our longitudinal sample data collection, continues to be a differentiator of our models and increases power for discovering true meaningful relationships.

### TO2 Assessing Applicability of Prebiotic Dosing and Postbiotic Output In Vitro

Commercializing our prebiotics for ceramide requires consistent and repeatable effects across a host of diverse facial microbiomes. Our targeted prebiotics for ceramide postbiotics must not harm the skin microbiome community members needed to produce those postbiotic ceramides. Our in-silico work has shown that metabolisms involved in ceramide production are redundant, and although we have already completed an in vitro proof of concept on a small number of empirical microbiome cultures (N=10), we need to confirm that product-relevant concentrations of target prebiotics and postbiotics are applicable to a larger number of microbiome communities derived from the diverse population of samples collected in TO1. We use toxicity and viability studies to examine this applicability, repeatability, and dosing. From these experiments we also measure postbiotic ceramide production while subsampling for metagenomics and metabolomics. We examine the commonality of ceramide metabolisms, predict and examine metabolic shifts and create models, using metagenomics and metabolomics.

### In Vitro Experiments to Assess Effects of Compounds Across Diverse Microbiomes

Growth curves of each empirically derived microbiome in LB growth media is the easiest and quickest way to assess bacterial growth in the presence of various concentrations and dosing of prebiotics and postbiotics. This method allows for the assessment of growth defects of a bacterial community if compound concentrations are so high that they reduce cell doubling times compared to untreated cultures. We will also subsample a set of these experiments for metagenomic and metabolomics studies to examine the community composition and functional shifts induced by our target prebiotics and postbiotics in culture over time. Diversity measures from TO1 are used to select subsample sets.

### Growth Curve Experiments:

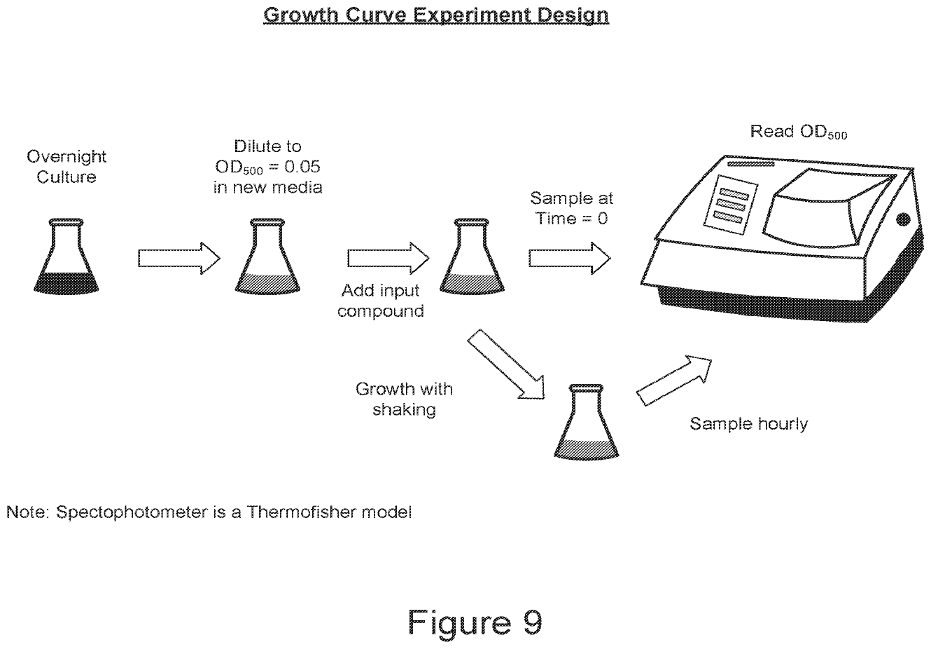

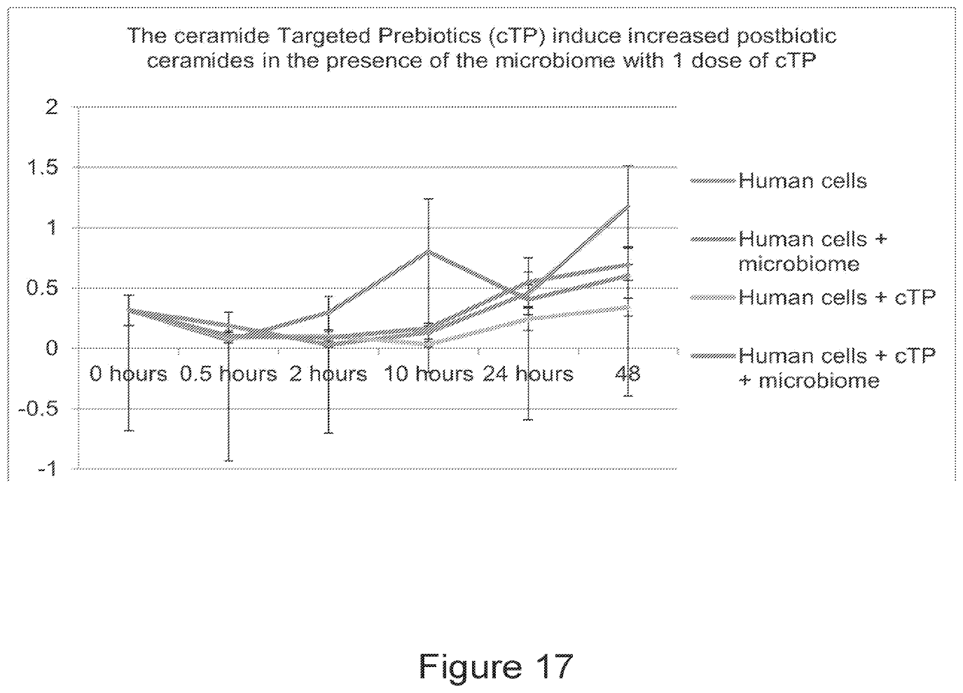

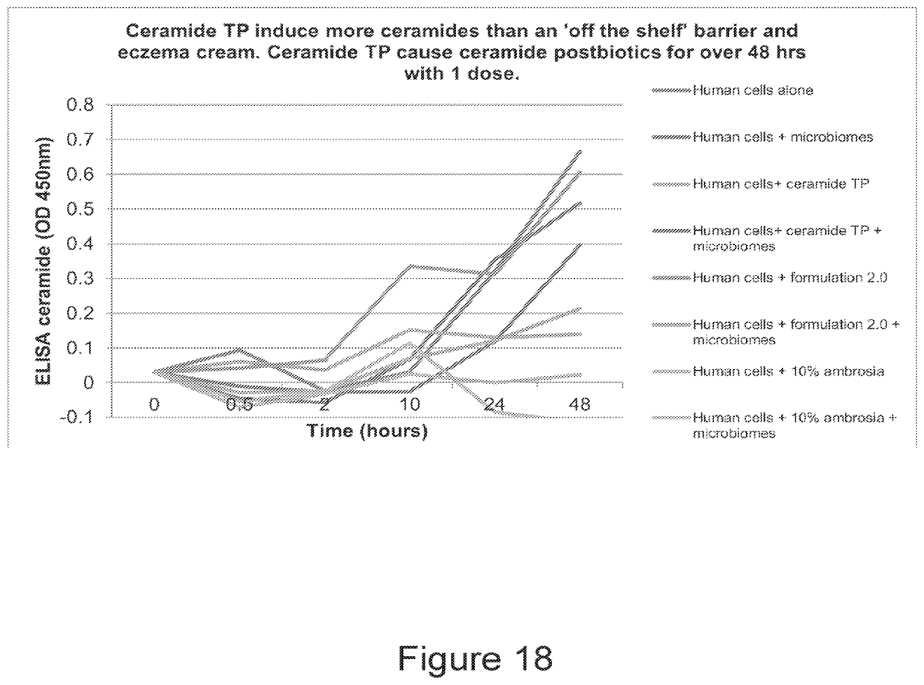

In order to perform growth experiments, empirical cultures grown overnight in LB broth are back diluted into new LB broth with varying concentrations of each compound so that the starting optical density (OD) at 600 nm is 0.05. Cultures are normally grown for 5 hours with shaking at 37° C., and samples taken for an OD₆₀₀ reading as shown in FIG. 9. Longer growth experiments were also completed to examine the time of postbiotic production from 1 dose of TP as shown in FIGS. 17 and 18 as examples Postbiotic Repellent compound is produced for at least 3 hours after the addition of the iTP. FIG. 16 shows an example of postbiotic production after addition of Targeted Prebiotic. This is one demonstration of a predicted prebiotic input compound spiked into a mixed empirical skin culture sampled post-spike at 3 hrs for GCMS. The repellent output compound was found to be at levels that are higher than are necessary to result in repellency for Anopheles gambiae.

### Viability Experiments Measure Toxicity of Compounds Across Diverse Microbiomes:

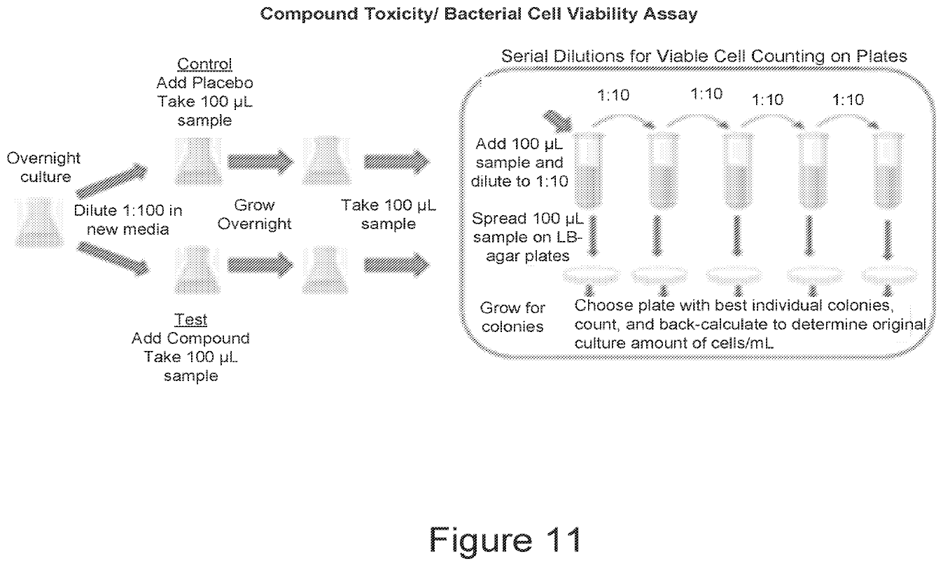

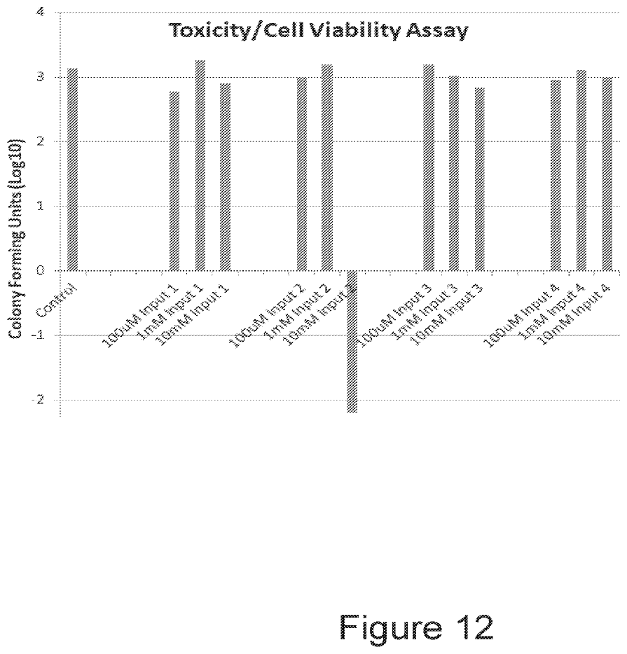

A spike experiment for viability of the cultures (TO1) was designed in our previous work to measure longer term toxicity effects of target prebiotic and postbiotic ceramides on the microbiome. Here we scale up our methods to plates (FIG. 11). These experiments are performed by diluting an overnight culture 1:100 in fresh liquid media containing specific concentrations of target prebiotics, ceramide postbiotics, or other formulation ingredients. Samples are grown overnight then 100 μL sample is taken at 0 and 16 hours. After plating on nutrient agar plates colony growth is quantified. FIG. 12 shows tests across several predicted TP at different concentrations for microbiome health and viability.

### Examined Prebiotic and Postbiotic Ceramide Production Across Diverse Microbiome Collection.

After examining tolerances for our target prebiotics for ceramide postbiotics, we measure the actual postbiotic production in vitro. In our work, we had variability in postbiotic output values (within 10-15% range) when studying our postbiotic mosquito repellent. We have yet to measure this variability across a diverse set of microbiomes in vitro for our prebiotics and ceramide postbiotics. We subsample each growth and spike culture experiment and use enzyme-linked immunosorbent assay (ELISA), for postbiotic ceramide detection.

### ELISA Detection of Ceramide Prebiotics and Postbiotics:

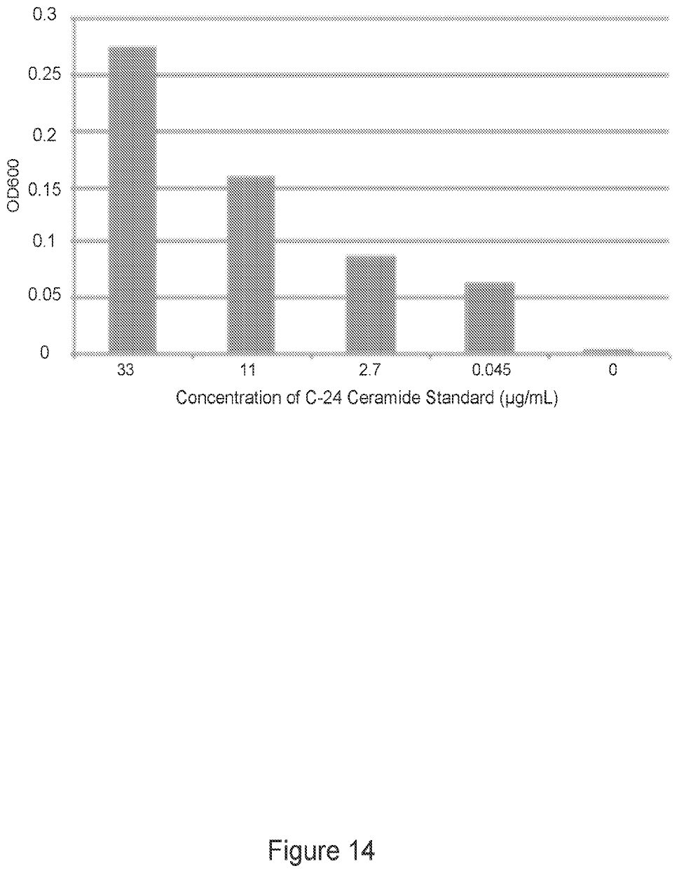

In our older work we successfully developed an in-house ceramide ELISA to provide for optimal detection from tissue culture media, bacterial growth media, and cell pellets of either human or bacterial origin. In order to prepare samples for this ELISA, we utilize the Folch method for lipid isolation. An overview is included here: the final dried sample from an experiment is resuspended in 200 ul of methanol. We add 100 ul of each resuspended sample in duplicate to a 96 well plate and incubate overnight at 4 C. The next day the plates are allowed to air dry in a hood until all methanol has evaporated. Blocking buffer consisting of phosphate buffered saline (PBS) plus 3% (w/v) nonfat milk is added for 2 hours at room temperature with rocking. The blocking buffer is removed and 100 μL of new blocking buffer containing 1:100 mouse IgM anti-human ceramide C-24 antibody is added to each well. After incubation overnight at 4° C. with rocking the plates are washed 5× with 300 μL of PBS plus 0.05% tween-20. 100 μL of goat IgG anti-mouse IgM conjugated with horseradish peroxidase in PBS plus 3% bovine serum albumen (BSA) is added to each well, and incubated at room temperature (˜22 C) with rocking for 2 hours. Wells of the plate are again washed 5× with PBS plus 0.05% tween-20. At this point 1× TMB (3,3′,5,5′-Tetramethylbenzidine) and 1×TMB reaction stop solution are used to produce a colorimetric product quantitatively read on a plate reader at OD₄₅₀. The result is then compared to a known standard curve generated (FIG. 14) using a serial dilution of ceramide C-24 to determine ceramide amounts of each sample. FIG. 17 shows an example of the inducement of postbiotic ceramides by the cTP.

Examined dosing across microbial metabolomes using metagenomics and metabolomics:

### Subsampling Scheme:

Since we are completing many laboratory experiments (for example N=102 at minimum excluding controls and duplicates and assuming we use only our current samples at a single dosing amount), we create a subsampling scheme for metagenomics and metabolomics. Cultures or other samples with an associated original skin sequencing diversity assessment (as beta and alpha diversity and Bray-Curtis beta diversity metrics, calculated from the filtered OTU tables), that are of low, medium or high microbial diversity based on k-means and hierarchical clustering are chosen aiming for 5 samples in each category. 30 paired samples, 15 treated and 15 untreated, are subsampled for shotgun sequencing and metabolomics. 10 μL of culture is aliquoted into a 100 μL tube and stored at −80 C until processed. We then follow methods described in TO1 for extraction, library preparation, sequencing, QC, annotation, and metabolomics. Additional platform methods are explained here.

### Platform Methods and Additional Analysis:

As was done in our previous work, we determine metabolite scores driving differences between microbial communities in our cultures from metagenomic shotgun data. We specifically focus on ceramide pathways and examine other off-target pathways that appear more regulated. We note that small scale work and that presented in our older work has shown that our prebiotics for postbiotic ceramides—as well as other mid-stage metabolites are very targeted.

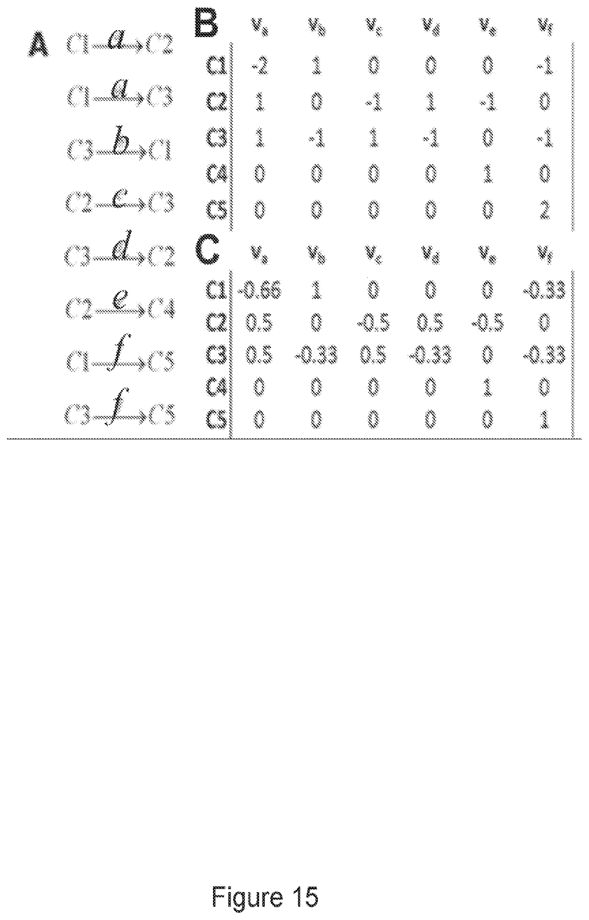

We have streamlined these data processes into an internal pipeline for analysis. Here we briefly explain the methods in the pipeline to examine in silico metabolic processes based on metagenomic sequencing (FIG. 15). We calculate the metabolome compound community M, from KEGG. We calculate G, the matrix or vector containing the counts of genes related to processes directly from the Enzyme Commission (EC) number sample abundances found in the annotated metagenome data rather than a predicted metagenome dataset as in the preliminary data. G is quantile and log 2 normalized and then multiplied by M to get a weighted score of predicted turnover per metabolite (also see FIG. 15 item c). We use methods such as PCoA to explore discrimination between sets of ceramide metabolites in the sensitive and non-sensitive phenotypes. We also calculate within sample organism MetCon scores, but with several differences as described here. G is multiplied by the matrix of per sample, per organism fractional probabilities of abundance, prior to multiplication by M. Annotated gene abundances per sample per organism are found using the assembled metagenome sequence data. This results in a per sample per organism MetCon score. The top 10% of scores are compared to the top 10% scores generated from within organism sample metabolite scores.

For comparisons of sample scores, Kruskal-Wallis rank sum tests are used. Lastly, we also assess stability and consistency of the core organisms and functions present in each culture (and in turn dosing) across phenotypes (sensitive (N=17) vs nonsensitive (N=34) and demographics from lab cultures. To assess this stability and consistency of MetCon scores, across individuals for phenotype we will use statistical tests such as Mantel tests⁵⁰ and Procrustes Analysis⁵¹. We analyze microbiome community dynamics with the addition of our proposed target prebiotics and postbiotics. This technical objective is to observe community shift as it relates to species that may harbor ceramide related pathways. So far we have not found that the presence of certain target prebiotics and/or the production of postbiotics results in certain microbiome species outcompeting or alternatively declining. Part of our platform examines any unintended community shifts after the addition of the prebiotic which may adversely affect the skin health and demonstrates safety. Here we also create in silico models for the microbiome and metabolic inputs and outputs validating with metabolomics—which aids in narrowing the parameter space for TO3, TO4, TO5, and our biochemistry platform. We describe the feedback between our biochemistry and bioinformatic models below.

Having developed appropriate extraction methods to examine the predicted postbiotic output compounds for measurement, we used in vitro experiments (such as FIG. 16) and GC-MS, ELISA or antibody tests, to assess if fueling of specific metabolic pathways using our predicted target prebiotic input compounds would produce endogenous postbiotic compounds at efficacious levels. We used our single and mixed community in vitro cultures grown with the predicted prebiotic candidate input nutrients as identified in the preliminary metagenomics study. To demonstrate that the prebiotic inputs are indeed inducing the postbiotic outputs, we performed spike-in experiments (see FIG. 11), in which an overnight culture was spiked with prebiotic inputs, grown for several hours, and were then analyzed analytically for example by GC-MS, ELISA, or antibody detection.

Initial screening of prebiotics and postbiotics to establish concentrations using our platform show concentrations and safety that do not inhibit bacterial growth or affect viability of the cultures.

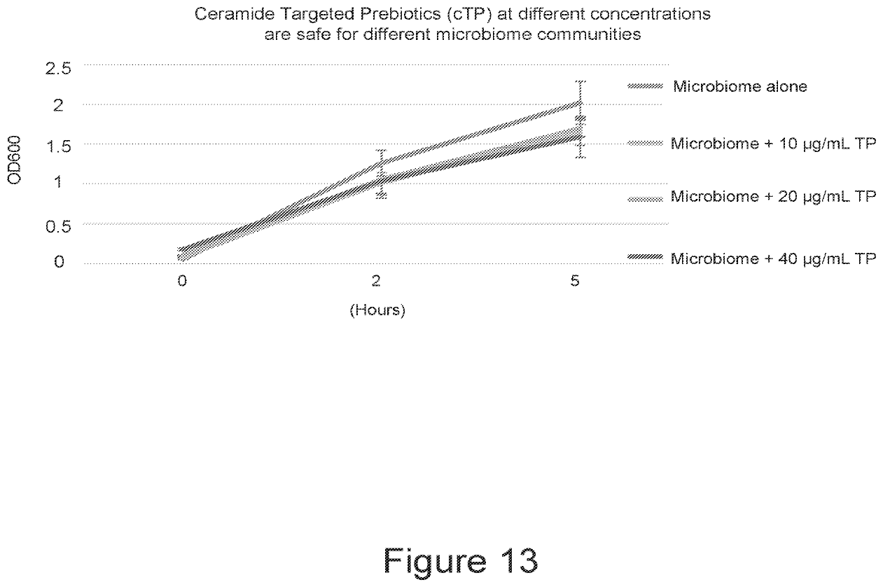

An example of an in vitro growth experiment is show in FIG. 13. Empirical samples will yield both a quantitative and qualitative variety of ceramide postbiotics due to differences in functional gene content of naturally occurring microbiome communities. How quickly communities respond may also vary, although we expect the ceramide composition and response times to be in a relatively tight range, 10-15%, as previously measured. We are confident that the methods validated in our previous study will mitigate the overall technical risk. It is possible that prebiotics or the postbiotic ceramides may cause a deleterious microbial community shift in some samples. If this is the case, then we will complete experiments on the long-term microbial community effects and their impact on the skin environment. We have also been able to correct these shifts with multiple dosing strategies. Since the ceramide pathways exist in sequencing data we expect that this will translate into production of ceramide postbiotics when the targeted prebiotics have been added in vitro. It is also possible our subsampling scheme is not powerful enough to detect a difference if the range of diversity across samples is too high. If this is the case, we will sample more experiments to enable testing across more microbiomes and metabolomes. We are aware that the in vitro conditions do not mimic the human skin environmental niche and might not result in the proper protein expression profiles needed to result in ceramide postbiotics from the prebiotics and thus we are prepared to modify our experiments as needed.

### TO3: Determining and Assessing Carrier Formulations for Prebiotics In Vitro and Ex Vivo

### Validated Formulation for Microbiome Health and Creation of Ceramide Postbiotics.

We described experiments earlier (TO2) that assessed validation of prebiotics across diverse microbiome cultures, here we examined the prebiotic in formulation or carrier compounds. Carriers give a cosmetically pleasing delivery system for the prebiotics: this is also known as the ‘formulation’. Factors that affect the carriers making up the formulation include hydrophobicity, pH, solubility, and long-term stability to maintain formulation efficacy. The carrier compounds in this case must not greatly shift microbiome health. We begin by choosing carriers that have passed a solubility and initial safety screening (derived from both safety data sheets and from the literature). These will include brontide, squalene, and glycerin, for example. We screened formulations at doses of prebiotics garnered from TO2 across our diverse culture collection. We perform growth curves and spike experiments to assess toxicity and viability as discussed in TO2 (FIG. 1&2). This set of experiments assured we do not have formulation toxicity that would cause growth defects or shifting in the skin microbiome. Results also inform how each formulation will affect the long-term health of diverse microbiomes.

From these growth and viability experiments we will examine if the formulations produce postbiotic ceramides. These data confirm the efficacy of both the delivery of the prebiotics, the production of the desired postbiotics, and safety. We do this again using an ELISA assay described in TO2, as well as subsampling experiments for metagenomics and metabolomics to examine in silico and pathway effects and off-target shifts (methods described in TO2). We also develop in silico models to evaluate carriers and final formulations for suitability with diverse skin microbiomes. The chosen carrier could affect the ability of a microbiome to induce a given pathway, resulting in no postbiotic production. These experiments also provide additional safety data. Thus TO3 along with TO4 allows us to scale up methods to assess carriers and develop in silico models to evaluate all future carriers.

### Models for Basic Formulation and Integration Between Biochemistry and Bioinformatic Platforms

Though the initial stage of our platform identifies prebiotics and their postbiotic compounds, formulations will need to be devised on a prebiotic-by-prebiotic basis. We scale and aid this process by developing in silico models from these in vitro (TO1,TO2, TO3) experiments that reduce the parameter space (e.g. dosing, timing, carriers) making in vitro and ex vivo and eventually in situ experiments more efficient and effective. From our in vitro formulation testing here we subsample again as per TO2 (N=30) samples for sequencing and metabolomics. We use data from metagenomics sequencing and metabolomics from these experiments (TO2) and here in TO3 to create models for the facial skin biome community that optimize the output of our postbiotic compounds.

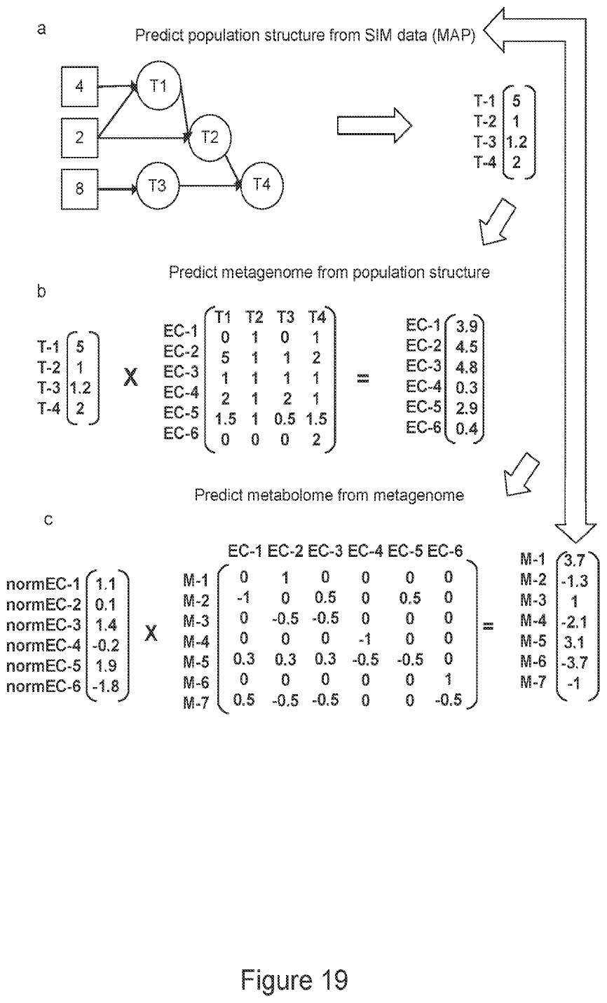

We develop in silico assemblage models that can be perturbed to examine changes in the community and its output metabolites and further developed here. The basic methods to develop the models here can only be represented from the data in context, as such we give examples of previous models to demonstrate the power of the methods (summarized here in brief). To create models 1) we use the set of key organisms garnered from the TO1 to create an interaction network: and step 2) represent this network as a set of explicit relationships inferred from the predicted compound data (garnered from TO2 and TO3) to create a predictive model. Step 1 is essentially the generation of a Bayesian inference network of microorganism assemblages as a directed cyclical graph (DAG) shown in FIG. 19 in which the parent nodes are changes in environmental parameters over time and space and the daughter nodes are changes in the relative abundance of the community. In this case the environmental parameters are the predicted metabolite compounds and their mass estimated from metabolomics (TO1, TO2, and TO3). Directed edges between nodes indicate correlations. Such a network can be generated with standard software that implements Bayesian network inference (such as the bayespy python package), based on parameters from the predicted compounds and organisms present in the metagenome data. At step 2, the value of the nodes needs to be expressed as a function of the value of its parent nodes. Again, this problem can be addressed by standard tools of unknown response surface learning, such as artificial neural network (ANN) tools, a form of artificial intelligence methods. These generated ANNs represent microbial community structure in terms of mathematical equations that best explain the data, and we use them to predict the relative abundance of taxa in time or space as functions of changing environmental conditions. These ANNs capture potentially causal relationships between the changing abundances of different taxa, although relationships between taxa could arise through taxon proxies for changes in environmental parameters. In this case the relationships are parameterized by the metabolomic results or other high throughput analytical method that captures biochemical changes. We constrain the model to ceramide compounds and the top 25 compounds from the metabolomics dataset for usability. These models parameterized by the early empirical data can help us to assess the consistency of the desired phenotype (for example atopic dematitis, sensitivity, or skin condition). Final models can be used to aid parameterization in other experiments (dosing, TO3, TO4, for example as a stand in for clinical populations), can be compared statistically to the results from LCMS, metabolomics, etc., experiments (TO3, TO4), decrease time and cost, as well as allow the ability to understand dosing/formulation to achieve a specific metabolomic profile (for example specific postbiotics desired).

Our prebiotics and carriers have safety data sheets (SDS) available that confirm the nonhazardous nature of each target prebiotic, carrier, and postbiotic compound. Given this we expect our newly designed formulations will most likely be safe as well. We have been using in silico models as described here to aid in our laboratory work and have found this to be accurate in previous experiments within 2-17.4% variability from empirical values. It might be expected that introducing more microbial community diversity (more samples from cultures developed from skin samples) into the system could make model fit more challenging and cause an inability to converge for models, however, focusing on core community functions associated with our prebiotic ceramide compound turnover, is another approach that has allowed convergence. A challenge to commercializing our platform has been the surprising effect that many “safe” compounds and products have on the skin microbiome. We have screened several preexisting facial skincare products and skincare carriers and have found they are not microbiome friendly and kill or shift existing host communities. Shifting communities can increase negative and pathogenic organisms that can reduce the protective skin barrier and reduce skin health. Our formulations are microbiome safe, as well as safe for humans. Many of these issues were resolved early on in our testing TO1 and TO2, where we determine maximum values of each ingredient that the microbiome can tolerate. These findings help us to determine a final formulation of carriers for our prebiotics.

### TO4: Assessing Formulation Dosing and Postbiotics Consistency Ex Vivo

Here we scale up our host-microbiome trans-well assay system to examine the carriers, dose and timing for formulation necessary for efficacious delivery of our prebiotics building on knowledge from TO1, TO2, and TO3. We use these assays in TO5 to evaluate and generate safety data.

Host-Microbiome Assays with Formulations

### Scale Up Host-Microbiome (Trans-Well) Assay

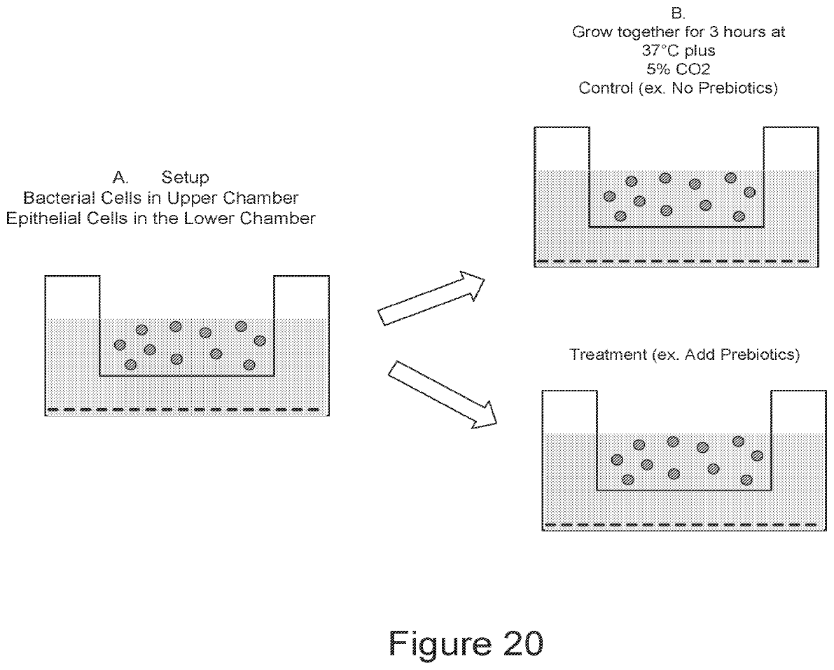

We have developed and validated a host-microbiome assay system (FIG. 20). This system allows human cells to grow in the presence of a microbiome culture. Here, we describe this method and scale up this assay. First, healthy and confluent adult human epithelial keratinocyte (HEKa) cells are passed and equally distributed between new 16-well tissue culture plates in Epilife media plus antibiotics plus HKGS (human keratinocytes growth supplement). Cells are grown at 37° C. plus 5% CO₂ until the wells are confluent. Empirical microbiome cultures are prepared in LB media overnight. The day of the experiment, the HEKa plates receive 1.5 ml of new Epilife media plus HKGS (without antibiotic). Each well then receives a trans well insert with a 0.4 um membrane. The overnight microbiome culture cells are spun down, the media is removed, and the pellet is resuspended in Epilife plus HKGS so that the final OD600=1.0. 500 ul of this mixture is added to the insert portion of the trans well. Treatments of prebiotics and carrier are then added. The experiment is incubated for 48 hours while samples are taken at times 0, 0.5, 2, 10, 24, and 48 hours. In order to scale up, we use a 12 well format plate with inserts in order to double the speed at which we can experiment and gather data for analysis.

Collection and Extraction of Ceramides and Associated Lipids from Host-Microbiome Experiments for Analysis

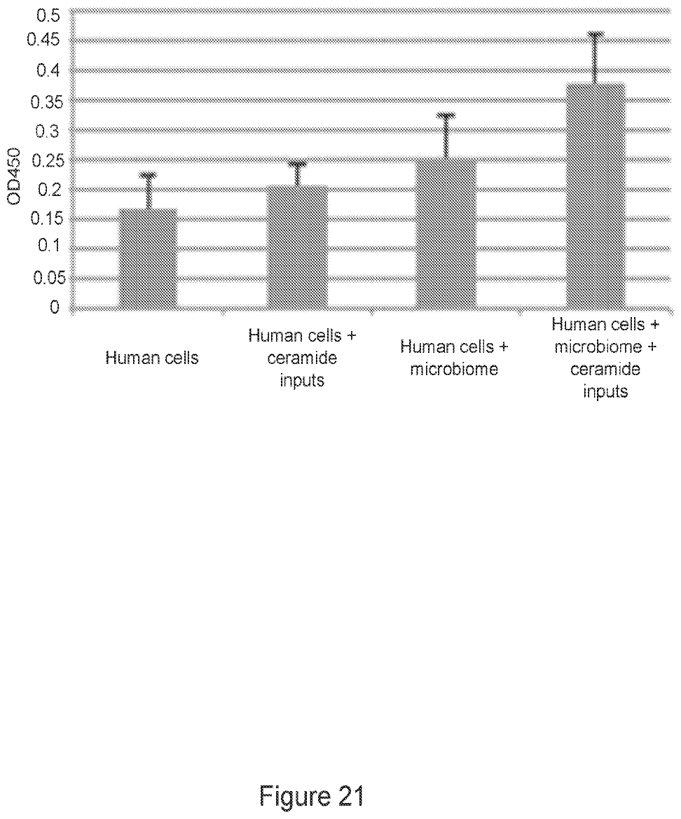

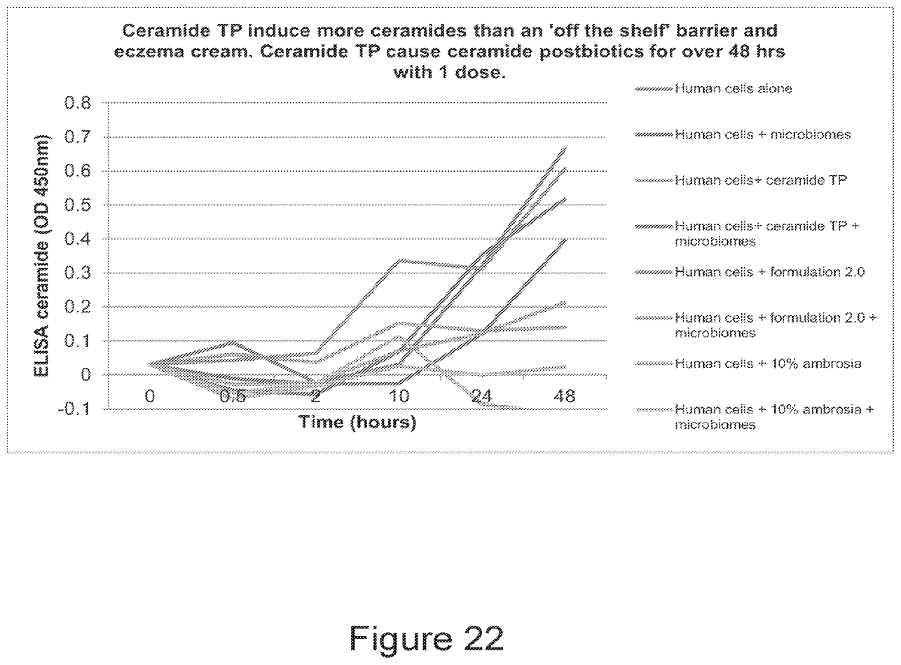

During the course of the host-microbiome experiments, wells are harvested for both supernatants and for cells (FIGS. 20, 21, 23), which can then be analyzed in downstream experiments for various purposes (see TO5). The supernatant is removed first for additional analyses, 1 ml of phosphate buffered saline (PBS) is added to each well and then cells are mechanically removed. The PBS/cell mixture is spun down at 2000 g for 5 minutes and the supernatant is removed leaving the cell pellet for later analysis. We examine both cells and supernatants for ceramides. Cell pellets are extracted via the Folch method. Postbiotic ceramides in the supernatants are also harvested similarly, except the supernatant is substituted for water in the initial Folch ratio (3chloroform:1 methanol:1 water).

One example of measuring postbiotic ceramide production is given in TO2 via ELISA. We also subsample supernatants for metagenome and metabolome analyses in silico using our subsampling scheme (N=30, 15 treated and untreated pairs).

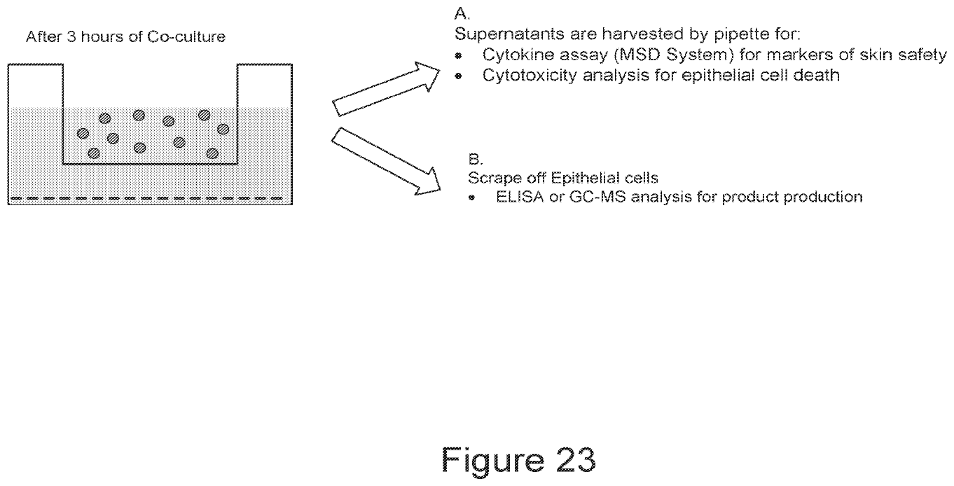

We have already performed several initial experiments to determine the feasibility and reliability of our host-microbiome approach (FIGS. 20 and 23 and for example 18, 21 and 22). Using these methods, FIG. 18 shows the production of ceramides from cTP with different carriers, and how the cTP or BioBloom™ containing formulations are better at producing ceramides than an off the shelf barrier cream (“ambrosia”). Further, these TP cause the production of ceramides for more than 48 hrs.

These novel experiments have yielded strong insights into the reliability and repeatability of our prebiotics to enhance the concentration of ceramides ex vivo. We are aware that the interaction of the microbiome culture with tissue culture cells in a liquid media can be problematic. Tissue culture cells are known to be fragile under the best of conditions. However, here we examine the changes of ceramides in the skin cells and the media rather than their overall health. Additionally we have completed assays examining shifting amounts exchanged growth media and dosing across time to find an optimal exchange based on the human cell type (data not shown). We can also employ a layered skin tissue approach with culture media.

### G2TO5 Assess Markers of Safety Using Ex Vivo Host-Microbiome System

Here we use our scaled-up host-microbiome trans-well assays (TO4) to assess markers for safety including irritation, inflammation, sensitivity, cell health, and cell death. These results build safety data for our ceramide prebiotics, and the methods give as examples here could be used for other candidate TP.

### Collection of Ex Vivo Samples for Examining Markers of Safety:

Supernatant and cells are collected post host-microbiome trans-well assay. At the time of collection for each time point sample, the trans-well insert containing the microbiome sample is removed and discarded, and 2×200 ul aliquots of supernatant from the remaining human keratinocyte side of the well are transferred to microcentrifuge tubes and frozen until use in the two assays (FIG. 23).

### Evaluating Irritation, Inflammation, and Sensitivity Using Ex Vivo Assays

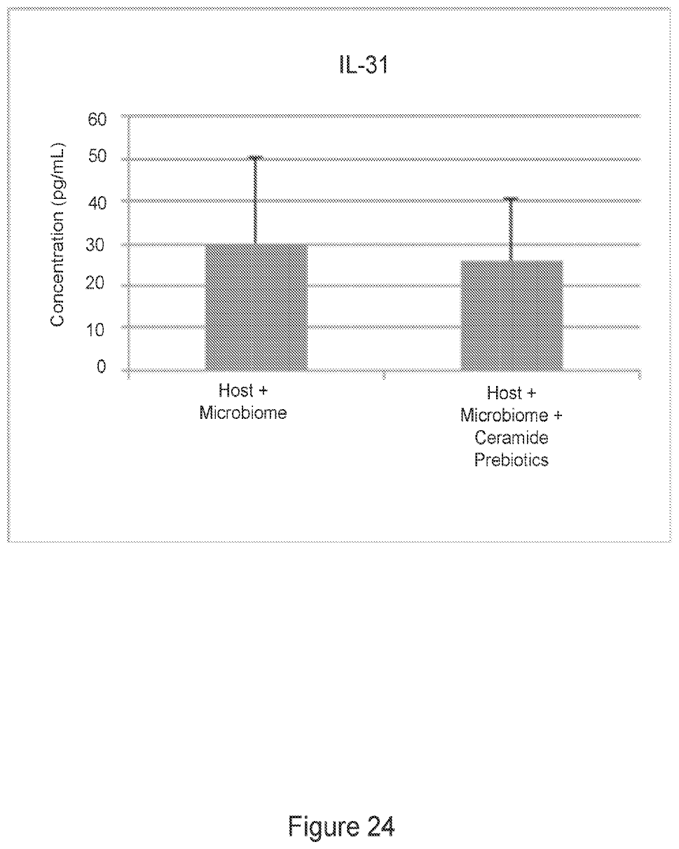

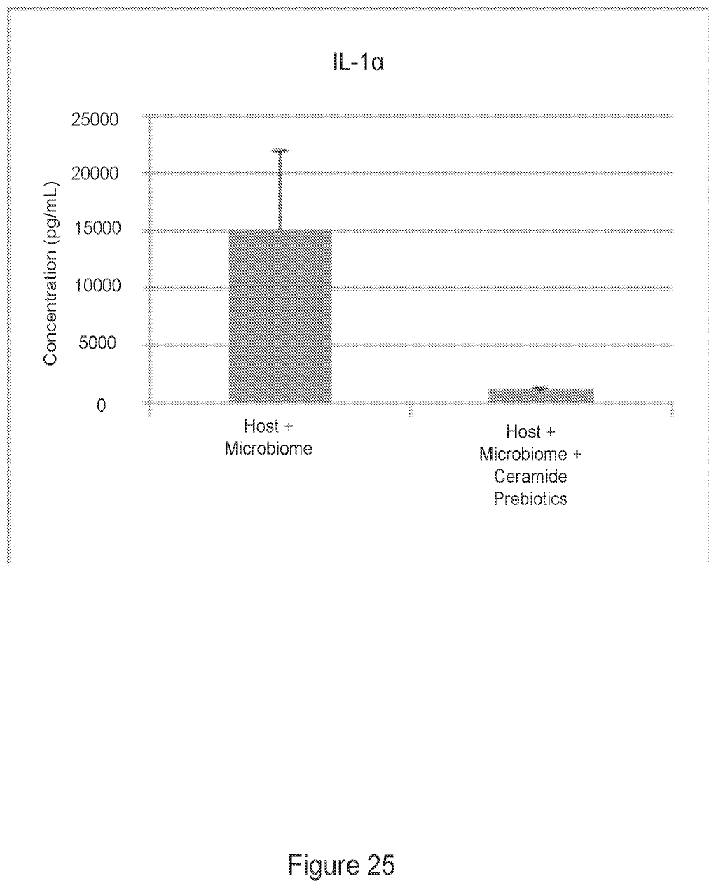

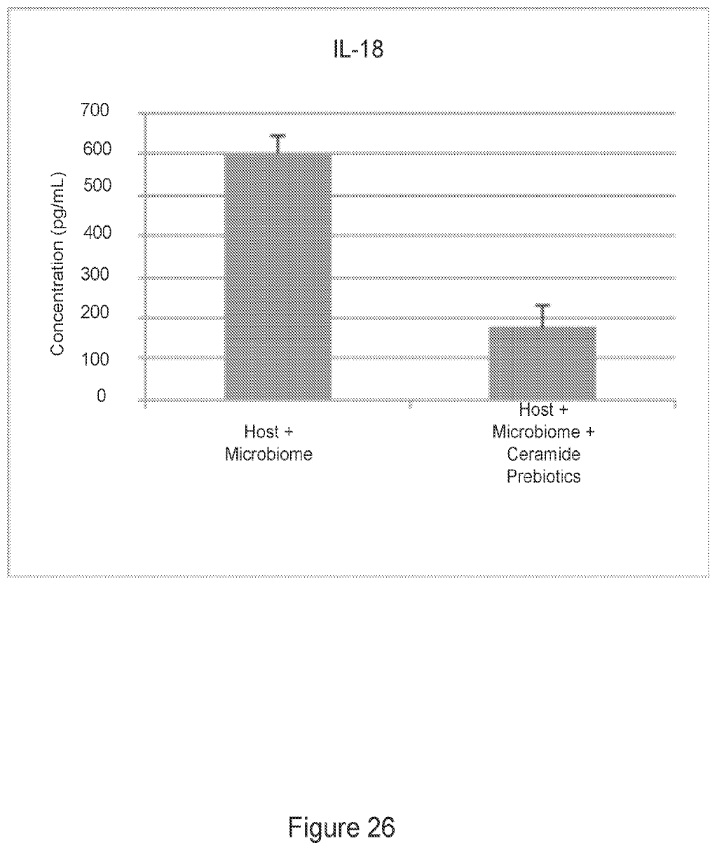

In order to evaluate irritation, inflammation, and sensitivity—all related to skin safety, we examine common skin inflammatory markers. For this we have currently been using our human-microbiome trans well system (described in TO4) and take supernatant samples for use in a multiplex cytokine ELISA based system (MesoScale Discovery [MSD], Rockville, MD). This MSD system allows for up to ten customizable cytokine target antibodies set up in a high-throughput 96-well format. With results shown in FIGS. 24-26, we use cytokines IL-8 (a marker of irritation and sensitization), IL-1a (a marker of irritation and sensitization and skin barrier maturation), IL-18 (irritation and contact allergies), IL-31 (trans epidermal water loss), and TNF-alpha (skin barrier formation).

FIGS. 24-26 illustrate results of cytokine markers show that ceramide Targeted Prebiotics reduce markers of sensitivity, irritation in the host-microbiome (trans-well system). Prior to each assay, 3 microbiome community cultures were used and experiments were completed in triplicate.

After prepping the MSD cytokine plate, supernatant trans-well samples taken during preselected times during experiments are added to the wells. The plates are then run in the MSD detection machine, which can detect picogram amounts of the cytokines. We generate a standard curve from known concentrations of each cytokine to calculate quantitative cytokine concentrations from our experiments. We have examined quantitative differences in cytokine concentrations between host cell alone, host and microbiome, and host-microbiome and prebiotic for postbiotic ceramide production.

### Evaluating Ex Vivo Trans-Well Assays for Cell Death

In addition to the cytokine markers of safety and cell health, we evaluate cell death from the host-microbiome assays (TO4) using the Cytotox 96 Cytotoxicity Assay (Promega Corp. Madison, WI). This plate-based assay detects the extracellular activity of lactate dehydrogenase (LDH), which is a cytosolic enzyme in healthy cells but is also released during cell lysis. Released LDH, indicating cellular death, turns tetrazolium salt into a red formazan product, which can be measured using a plate reader. We will collect supernatant samples during the host-microbiome experiments (TO4) to examine both microbiome and formulation cytotoxicity on the health of the human keratinocytes (host) cells in each sample plate well. Complete cell death by a lysis reagent (kit provided) acts as our positive control, while an untreated well will be the negative control. We quantitively compare host cell death from our formulations with dosing garnered in TO3 and TO4 across the cultures. We note that we will baseline the microbiome cultures on their own and the supernatants to understand to complete a proper control set for each experiment.

We have baselined cytokine and cell health and death assays. We have tested several different microbiome community cultures in our host-microbiome assay and examined this set of cytokines, with initial data showing no serious safety issues. The same samples in the cell death assays also showed low increases in cytotoxicity, so we anticipate that more cultures with formulations will yield similar results. One major concern that we have with these assays and the samples generated from the host-microbiome assay system is that we do not know how each empirical microbiome culture will react with the human keratinocytes. While we have not had challenges with these methods, it is possible that some microbiome samples will produce some off-target metabolites that are detrimental to human cells while in culture media. We monitor the safety of these off-target effects by looking at metagenomics and metabolomics from the host-microbiome samples. While potentially harmful empirical samples may exist, redundancy across prior skin microbiome samples in silico for ceramide pathways gives us confidence that the majority experiments will yield results that demonstrate the symbiotic and beneficial nature of the naturally occurring host-microbiome interaction.

### G2TO6 Assessment of Postbiotic Ceramide Production on Human Facial Skin

Here we apply current formulations based on previous TOs to examine the skin microbiome's ability to produce postbiotics from the target prebiotics as well as to determine how long postbiotics exist on the skin surface (in vivo). These experiments will provide insight into both product safety as well as how often our product will need to be applied for optimal skin care maintenance.

### Basic Skin Reaction Assessment

We completed a 24-hour patch test in a small cohort (N=9) of trial volunteers from our ongoing study with the ‘ formulation’ (Beta 1.0 Study) (carrier TO3 and prebiotics dose based on previous discoveries TO4). Skin reaction tests are a common way to assess irritation and sensitization⁷⁵⁻⁷⁷. Briefly, 0.21 mL of the formulation was applied to a small nickel sized area on the forearm near the antecubital fossa. After 24 hours the area was self-assessed for any redness, irritation, or sensitization. This is important to do before using the formulation. No changes in redness, irritation, or sensitization were reported. Metabolomics results did not show any increases in markers of basic irritation.

### In Vivo Examination of Skin-Microbiome Production of Target Postbiotics

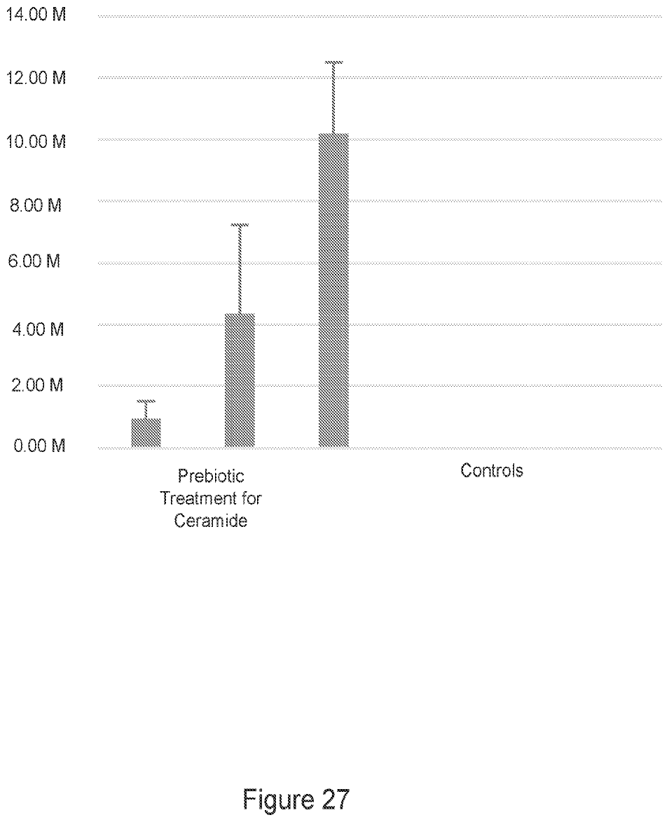

We validate postbiotic production using metabolomics. We have completed a small (N=3, 3 sites, duplicate) study to baseline the use of metabolomics to have the power to examine shifts due to the application of our prebiotics for ceramide postbiotic, prebiotics in ethanol at 1%, (FIG. 27). Additionally, we have assessed in situ production of ceramides on the skin by sampling swabs pretreated in EtOH (see TO1 for methods for metabolomic swab sampling from skin) and with a sorbent PDMS patch for approximately 8 hours over areas where the prebiotics were placed, as well as control samples with and without carriers. PDMS patches are extracted in EtOH and analyzed using GCMS. Absolute compound concentrations were determined by analyzing a dilution series of a standard with known compound amounts. Swabs are assessed via LCMS as per TO1. We compare skin swabs collected at the beginning of our clinical study to swabs and PDMS patches collected after this week-long 2× day use of the formulation. Swabs are collected as described in TO1.

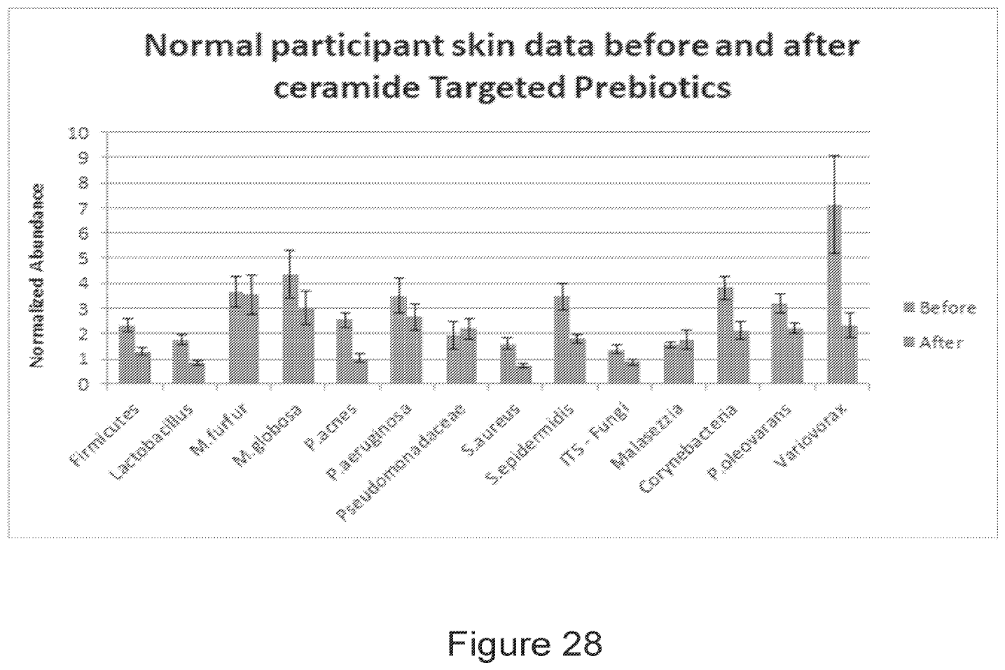

FIG. 28 shows abundances of several organisms from study participants' skin (n=42) before and after application of our cosmetic formulation with cTP, also known as BioBloom™. Participants were members of a 15 week IRB approved clinical trial.

### Potential Outcomes and Challenges:

We have completed a small metabolomics skin study for production of the ceramide postbiotics with 2 volunteers (3 sites, in duplicate) (FIG. 6). The ceramides are of extremely high molecular weight (MW). Here we expand the study to show the repeatability and applicability across human facial skin. We are aware there will be some variability in the postbiotic production, but our previous study showed that the variance was actually smaller than predicted from our in-silico models. We would expect similar dosing metrics regarding maximal output for our postbiotic, but it may be the case that we will have to adjust the timing due to differences in microbiome density as liquid cultures will have more bacteria than a dry flat surface like the skin. These data will continue to build a safety profile for our ingredients and formulations. We expect differences and variation due to spatial issues relating to the face patches and extrapolating from a liquid based culture system to a drier in vivo system. The patches and formulation carrier should help maintain a moist environment for the host-microbiome, but if we do not see the same amount of robust postbiotic production, then we may have to design some additional formulations. Further we know our in vitro cultures, ex vivo human-microbiome system, and in silico are imperfect models of a living system. However, despite the variation from these different methods, initial baselines and experiments have been within a very tight quantitative range (within 0.2-1.1%). Microbial niches can be extremely diverse, even in a small measurement space. However, since we focus on the microbial functional redundancy we have the power and potential to have a consistent postbiotic output across diverse human skin. We have strongly alleviated this concern by demonstrating with empirical evidence across human facial skins.

### G3TO7: Example 2: Using Our Platform-Validate Hyaluronic Acid as Another Microbiome-Generated Skincare Pathway and Develop a Carrier Formulation

Our second priority candidate is a prebiotic for hyaluronic acid. Hyaluronic acid is the most common ingredient in anti-aging cosmetics and a crucial ingredient in keeping moisture in the skin and promoting a healthy skin barrier. Hyaluronic acid can be found in variable length chains containing linked hyaluronic acid subunits. Here we assess the applicability and safety of our prebiotics for HA in vitro and ex vivo.

### Assessment of Empirical Microbiome Samples for Hyaluronic Acid Pathway Genes and Metabolites

GITO1 and GITO2, we will analyze the collected sequences for genes and metabolites that indicate the ability to produce hyaluronic acid as an end product from our target inputs.

### In Vitro Experiments to Assess Effects of Prebiotic Compounds for HA Across Diverse Microbiomes

We screen our culture collection for viability, toxicity, and growth experiments as described in TO2. This allows us to determine proper concentrations of target prebiotics that do not affect the health of empirical microbiomes. We aim to test ideal dosing and timing parameters by performing spike experiments with tolerable concentrations of target prebiotics. For the growth experiments we will take samples at times 0, 0.5, 2, 10, 24, and 48 hours. For dosing experiments, we will administer the first dose of treatment of our spike experiments at time 0 hours and again at time 3 hours.

### Harvesting and Detection of Hyaluronic Acid In Vitro

As hyaluronic acid is very soluble in water and thus various culture medias, we do not need to take extra steps to evaluate free floating hyaluronic acid levels produced by empirical microbiome samples. For detection of hyaluronic acid in liquid media, we use an ELISA detection kit.

### Selection of a Carrier for Formulation

As we have done in TO3, we assessed various carriers for proper suitability with the target prebiotics for hyaluronic acid. Again given the chemical properties of brontide, squalene, and glycerin we can use these again with HA. We will screen these formulations against our culture collection for viability, toxicity, and growth (microbiome health compatibility) and hyaluronic acid production via ELISA.

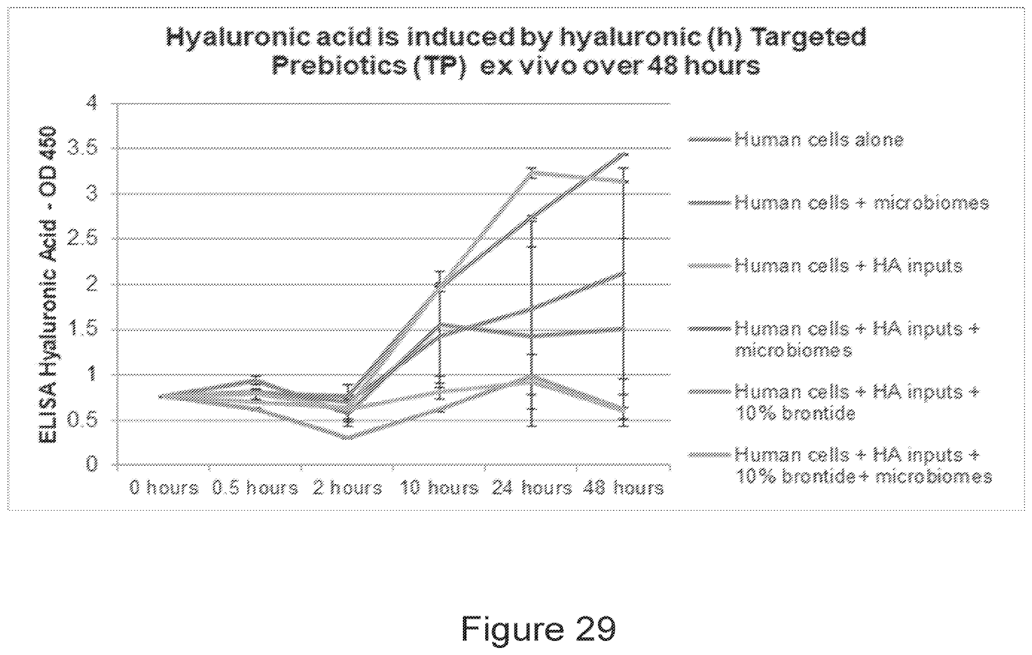

As shown in FIG. 29, the hyaluronic acid Targeted Prebiotics (hTP) induce increased postbiotic HA with the microbiome. Carriers affect resulting postbiotic production. Host-microbiome assays were conducted with 3 microbiome communities, where applicable. 1 dose at 0.02% of hTP was given for those applicable samples.

### Build in Silico Models for HA Production

We subsample these in vitro experiments for metagenomics (N=15) metabolomics (N=15) for each, as we have baselines for metagenomics and metabolomics cultures from TO3. Using methods described in TO3, we build in silico models of the assemblages and their functions. We parameterize relationships by the metabolomic results. We constrain the model to associated hyaluronic acid compounds and the top 25 compounds from the metabolomics dataset for usability. Again, these models, parameterized by the early empirical data can help us to understand parameters for our experiments.

### Potential Outcomes and Challenges:

Based on our early proof of concept from our earlier work—we anticipate hyaluronic acid production across our cultures. We expect that hyaluronic acid production at efficacious amounts (1-5%). Further, as hyaluronic acid is water soluble, we expect that formulation tests will be more straightforward and will provide a microbiome friendly environment. Despite early in vitro study efforts we have been unable to fully identify all of the genes and mechanisms involved in this process. We anticipate that a more comprehensive characterization will result from additional analytical power from sequencing and metabolomics work (TO1, TO6). Further, sequencing annotation on major databases are also lacking, however it is highly probable that a protein of homologous function exists, as we have already seen evidence of boosted hyaluronic acid in the presence of our prebiotic (for hyaluronic acid) in a small set of cultures.

G3TO8: Demonstration that Hyaluronic Acid is Produced in Both an Ex Vivo and In Vivo System

As we have done for our prebiotics for ceramides in TO3 through TO6, we examine production of our prebiotics to target hyaluronic acid in our ex vivo and on human skin to address critical questions of feasibility, safety, and efficaciousness of future hyaluronic acid prebiotic formulations.

### Evaluating the Ability of the Microbiome to Induce the Hyaluronic Acid Pathway in an Ex Vivo System

In order to determine if our target hyaluronic acid pathway promoted by the microbiome will be effective in vivo, we will first need to thoroughly test it with the formulations, microbiomes, and human epithelial cells. In order to do this, we will utilize the host-microbiome trans-well assay that we outlined in TO4. This assay will ensure that the human cells will obtain a higher concentration of hyaluronic acid as a result of the interaction of the microbiome in the presence of the target hyaluronic acid prebiotics formulation.

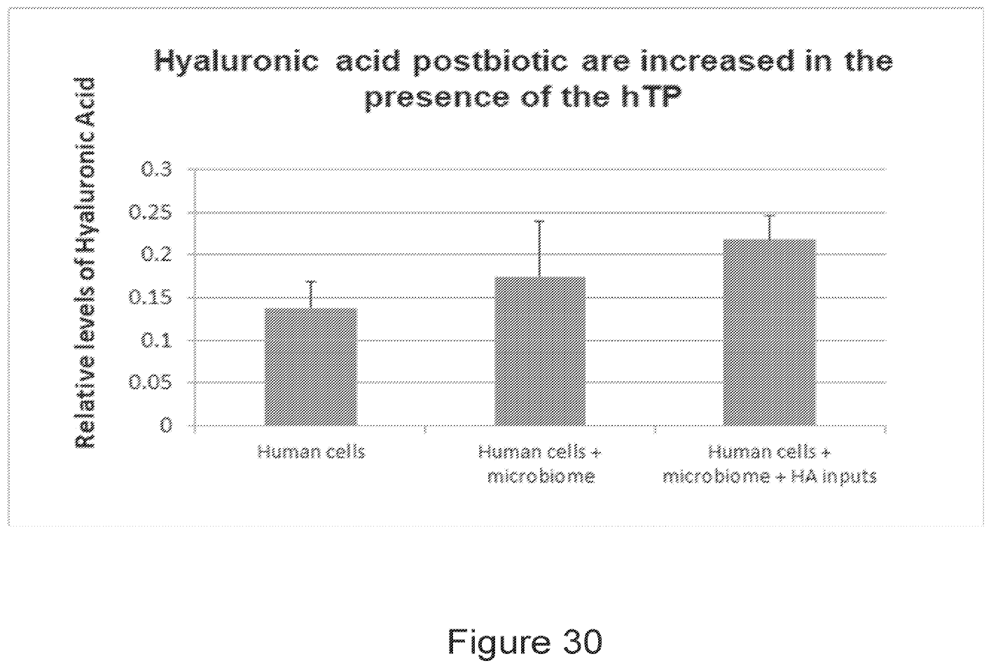

As shown in FIG. 30, host-microbiome (transwell) assays with hTP ELISA show increased HA postbiotics in the presence of the microbiome. All assays were done in triplicate and for those using microbiome communities, N=3 communities were tested (also each in triplicate).

### Testing for Sensitivity and Irritation Markers

Using experiments in TO5, we will examine how our prebiotic formulations for hyaluronic acid postbiotic affect the sensitivity, irritation, and overall health of human endothelial keratinocytes.

In Vivo Skin Testing for Microbiome Induced Hyaluronic Acid from Target Prebiotic Formulations

The last aim of this objective is to investigate for the production of hyaluronic acid in a consumer cohort and then to determine if this production will result in a positive skin health outcome. We also have evaluated Hyaluronic acid by GCMS samples derived from swabs and PDMS patch testing methods.

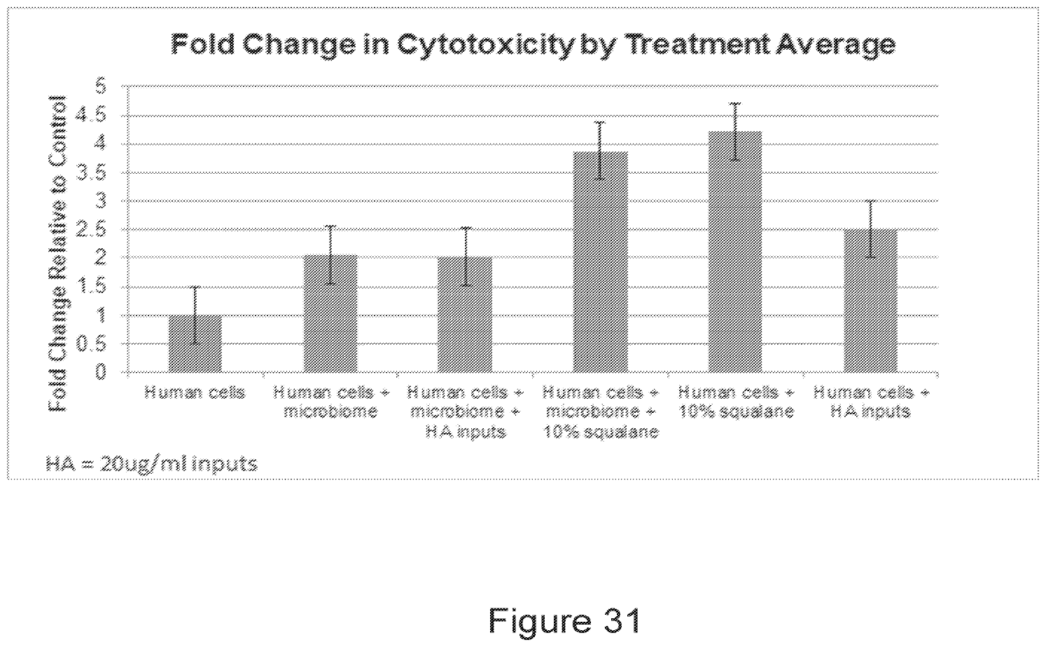

As shown in FIG. 31, ex vivo cytotoxicity experiments show that HA inputs (hTP) have less cellular toxicity than carriers or ingredients such as squalane. N=3 microbiome communities used where applicable, all experiments done in triplicate.

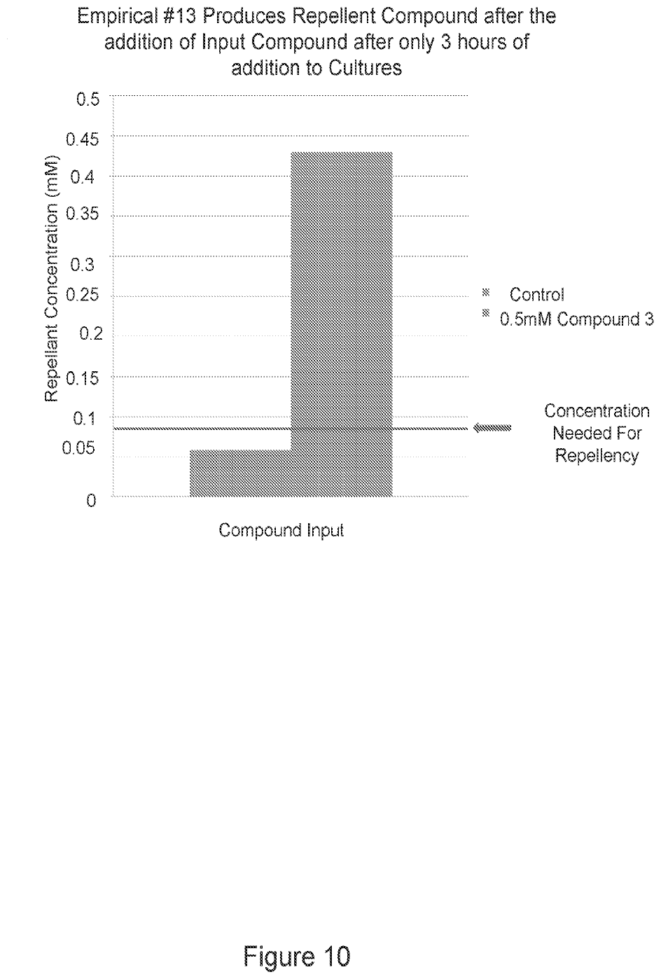

Example paradigm—using their induce the native skin microbiome to create targeted postbiotics, directly benefits human health and the environment. The targeted prebiotic solutions create postbiotics that are natural, super long-lasting, and efficacious. Further, from our first ingredient prebiotics for ceramides we have shown preliminary data for three high molecular weight ceramides being produced on the skin (FIGS. 10 and 27). Based on the literature, these specific ceramides have activity as known anti-melanoma compounds and in scar reduction.

To include these precision postbiotic compounds directly in a product would be cost prohibitive and technically infeasible to provide at scale using typical methods of production, but the example paradigm creates these compounds inexpensively and at efficacious levels. We change the economics of how we think about formulating products—and the compounds that are accessible to use. This represents a big technology jump in ingredients and products. Further our development of in silico models—allow us to examine the prebiotics in carriers—and their ultimate effect on the skin. Our long-term goal is to understand the human-microbiome skin system to the point of being able to engineer a product that maximizes its expression of beneficial compounds on the skin.

Further, the ability to stoke endogenous natural compounds through the in situ microbiome opens up an entire field to explore for additional human health and environmental benefits. Beyond the skin, the gut, and even in the environment, new products and processes could be made that access natural products in a way that is economically beneficial, and environmentally efficient and safe.

1. A method comprising:
obtaining, by a computing system including one or more computing devices that each include a processor and memory, sequencing data that includes a plurality of sequencing reads, the plurality of sequencing reads being derived from a plurality of samples;
aggregating, by the computing system, a number of individual sequencing reads of the plurality of sequencing reads to generate aggregate sequences, the aggregate sequences including one or more first sequences of the plurality of sequencing reads derived from a first sample of the plurality of samples obtained from a first individual and one or more second sequences of the plurality of sequencing reads derived from a second sample of the plurality of samples obtained from a second individual;
analyzing, by the computing system, one or more genomic regions that correspond to the one or more first sequences and the one or more second sequences to (i) determine one or more enzymes that correspond to the one or more genomic regions and (ii) determine one or more organisms having a respective genome that include the one or more genomic regions;
determining, by the computing system, a biochemical pathway that corresponds to an individual genomic region of the one or more genomic regions based on at least one enzyme of the one or more enzymes that corresponds to the individual genomic region, wherein the at least one enzyme activates a reaction related to the biochemical pathway;
determining, by the computing system, a number of compounds related to the biochemical pathway, the number of compounds including at least a first compound that is a reactant in the reaction of the biochemical pathway and a second compound that is a product in the reaction of the biochemical pathway;
determining, by the computing system, a first measure of a first amount of an enzyme of the one or more enzymes present in the first sample based on a number of the one or more first sequences that correspond to the individual genomic region; and
determining, by the computing system, that the reactant is a candidate prebiotic to treat one or more biological conditions present in one or more first individuals based on the first measure of the first amount of the enzyme.

2. The method of claim 1, comprising:
obtaining, by the computing system, analytical data obtained from the first sample by performing one or more mass spectrometry operations; and
determining, by the computing system, a first abundance of the reactant and a second abundance of the product in the first sample based on the analytical data;
wherein the reactant is determined to be a candidate prebiotic based on the first abundance of the reactant and the second abundance of the product in the first sample.

3. (canceled)

4. The method of claim 1, comprising:
obtaining, by the computing system, additional sequencing data that includes a plurality of additional sequencing reads, the plurality of additional sequencing reads being derived from a plurality of additional samples, the plurality of additional samples including first additional samples that correspond to a first set of environmental conditions and second additional samples that correspond to a second set of environmental conditions;
aggregating, by the computing system, a number of individual additional sequencing reads of the plurality of additional sequencing reads to generate additional aggregate sequences;
analyzing, by the computing system, the additional aggregate sequences to determine one or more additional genomic regions that correspond to the additional aggregate sequences; and
analyzing, by the computing system, the one or more additional genomic regions to (i) determine one or more additional enzymes that correspond to the one or more additional genomic regions and (ii) determine one or more additional organisms having a respective genome that includes the one or more additional genomic regions.

5.-17. (canceled)

18. The method of claim 1, wherein;
the first sample is obtained from skin of a first individual and the second sample is obtained from skin of a second individual;
the first individual is included in a first phenotype and the second individual is included in a second phenotype; and
the first phenotype corresponds to a presence of a biological condition with respect to individuals and the second phenotype corresponds to an absence of the biological condition with respect to individuals.

19. (canceled)

20. (canceled)

21. The method of claim 18, wherein the biological condition corresponds to an abnormality related to skin of individuals.

22. A system comprising:
one or more hardware processors; and
one or more computer-readable storage media including computer-readable instructions that, when executed by the one or more hardware processors, perform operations comprising:
obtaining sequencing data that includes a plurality of sequencing reads, the plurality of sequencing reads being derived from a plurality of samples;
aggregating a number of individual sequencing reads of the plurality of sequencing reads to generate aggregate sequences, the aggregate sequences including one or more first sequences of the plurality of sequencing reads derived from a first sample of the plurality of samples obtained from a first individual and one or more second sequences of the plurality of sequencing reads derived from a second sample of the plurality of samples obtained from a second individual;
analyzing one or more genomic regions that correspond to the one or more first sequences and the one or more second sequences to (i) determine one or more enzymes that correspond to the one or more genomic regions and (ii) determine one or more organisms having a respective genome that include the one or more genomic regions;
determining a biochemical pathway that corresponds to an individual genomic region of the one or more genomic regions based on at least one enzyme of the one or more enzymes that corresponds to the individual genomic region, wherein the at least one enzyme activates a reaction related to the biochemical pathway;
determining a number of compounds related to the biochemical pathway, the number of compounds including at least a first compound that is a reactant in the reaction of the biochemical pathway and a second compound that is a product in the reaction of the biochemical pathway;
determining a first measure of a first amount of an enzyme of the one or more enzymes present in the first sample based on a number of the one or more first sequences that correspond to the individual genomic region; and
determining that the reactant is a candidate prebiotic to treat one or more biological conditions present in one or more first individuals based on the first measure of the first amount of the enzyme.

23. (canceled)

24. (canceled)

25. The system of claim 22, wherein the one or more computer-readable storage media include additional computer-readable instructions that, when executed by the one or more hardware processors, perform additional operations comprising:
obtaining additional sequencing data that includes a plurality of additional sequencing reads, the plurality of additional sequencing reads being derived from a plurality of additional samples, the plurality of additional samples including first additional samples that correspond to a first set of environmental conditions and second additional samples that correspond to a second set of environmental conditions;
aggregating a number of individual additional sequencing reads of the plurality of additional sequencing reads to generate additional aggregate sequences;
analyzing the additional aggregate sequences to determine one or more additional genomic regions that correspond to the additional aggregate sequences; and
analyzing the one or more additional genomic regions to (i) determine one or more additional enzymes that correspond to the one or more additional genomic regions and (ii) determine one or more additional organisms having a respective genome that includes the one or more additional genomic regions.

26. The system of claim 25, wherein the one or more computer-readable storage media include additional computer-readable instructions that, when executed by the one or more hardware processors, perform additional operations comprising:
determining, based on the additional aggregate sequences, first amounts of first enzymes present in a first additional sample;
determining, based on the additional aggregate sequences, second amounts of the first enzymes present in a second additional sample; and
determining one or more differences between the first amounts and the second amounts.

27. The system of claim 26, wherein the one or more computer-readable storage media include additional computer-readable instructions that, when executed by the one or more hardware processors, perform additional operations comprising:
obtaining first additional analytical data obtained from the first additional sample;
obtaining second additional analytical data obtained from the second additional sample;
determining, based on the first additional analytical data, a first additional abundance of the reactant and a first additional abundance of the product;
determining, based on the second additional analytical data, a second additional abundance of the reactant and a second additional abundance of the product;
determining one or more first differences between the first additional abundance of the reactant and the second additional abundance of the reactant; and
determining one or more second differences between the first additional abundance of the product and the second additional abundance of the product.

28. The system of claim 27, wherein the one or more computer-readable storage media include additional computer-readable instructions that, when executed by the one or more hardware processors, perform additional operations comprising:
determining, based on the aggregate sequences, a plurality of organisms present in the first sample and the second sample; and
determining a subgroup of organisms included in the plurality of organisms.

29. The system of claim 28, wherein the one or more computer-readable storage media include additional computer-readable instructions that, when executed by the one or more hardware processors, perform additional operations comprising:
obtaining first additional analytical data derived from the first additional sample;
determining, based on the first additional analytical data, first additional measures of abundance for the subgroup of organisms in the first additional sample, individual first additional measures of abundance corresponding to a respective first measure of abundance for an individual organism included in the subgroup of organisms;
obtaining second additional analytical data derived from the second additional sample;
determining, based on the second additional analytical data, second additional measures of abundance for the subgroup of organisms in the second additional sample, individual second additional measures of abundance corresponding to a respective second measure of abundance for an individual organism included in the subgroup of organisms; and
determining one or more differences between at least a portion of the first additional measures of abundance and at least a portion of the second additional measures of abundance.

30. The system of claim 29, wherein the one or more computer-readable storage media include additional computer-readable instructions that, when executed by the one or more hardware processors, perform additional operations comprising:
determining one or more correlations between (i) at least one of the one or more first differences the one or more first differences between the first additional abundance of the reactant and the second additional abundance of the reactant or the one or more second differences between the first additional abundance of the product and the second additional abundance of the product and (ii) the one or more differences between at least a portion of the first additional measures of abundance and at least a portion of the second additional measures of abundance.

31. The system of claim 30, wherein the one or more correlations are determined using one or more Bayesian network techniques.

32. The system of claim 30, wherein:
the first additional sample is collected from a first environment that comprises a first formulation, the first formulation comprising a first amount of the reactant and a first carrier substance for the reactant; and
the second additional sample is collected from a second environment that comprises a second formulation, the second formulation comprising a second amount of the reactant and a second carrier substance for the reactant.

33. The system of claim 32, wherein the first amount of the reactant is different from the second amount of the reactant.

34. The system of claim 32, wherein the first carrier substance for the reactant is different from the second carrier substance for the reactant.

35. The system of claim 32, wherein the one or more computer-readable storage media include additional computer-readable instructions that, when executed by the one or more hardware processors, perform additional operations comprising:
determining one or more functions that determine abundances of the subgroup of organisms, wherein the one or more functions are determined based on (a) the first formulation and the second formulation and (b) the one or more differences between (i) at least one of the one or more first differences the one or more first differences between the first additional abundance of the reactant and the second additional abundance of the reactant or the one or more second differences between the first additional abundance of the product and the second additional abundance of the product and (ii) the one or more differences between at least a portion of the first additional measures of abundance and at least a portion of the second additional measures of abundance.

36. The system of claim 35, wherein the one or more computer-readable storage media include additional computer-readable instructions that, when executed by the one or more hardware processors, perform additional operations comprising:
generating a model that implements the one or more functions, the model having a number of parameters that correspond to conditions within the first environment and the second environment.

37. The system of claim 36, wherein the one or more computer-readable storage media include additional computer-readable instructions that, when executed by the one or more hardware processors, perform additional operations comprising:
obtaining values of the conditions that correspond to the number of parameters, at least a portion of the values of the conditions being different from additional values of the conditions that correspond to the first environment and the second environment; and
executing the model to determine abundances of at least a portion of the organisms included in the subgroup of organisms, wherein the abundances correspond to the values of the conditions.

38. The system of claim 36, wherein the model is generated using one or more artificial neural networks.

39.-42. (canceled)
